(19) United States
(12) Patent Application Publication
(10) Pub. No.: US 2016/0166719 A1
(43) Pub. Date: Jun. 16, 2016

(54) THERMAL BACTERIAL DECOLONIZATION

(71) Applicant: EMPIRE TECHNOLOGIES DEVELOPMENT LLC, Wilmington, DE (US)

(72) Inventor: Kevin Anthony Pezzi, Thompsonville, MI (US)

(21) Appl. No.: 14/418,871

(22) PCT Filed: Sep. 3, 2013
(86) PCT No.: PCT/US13/57850

§ 371 Date: Jan. 30, 2015

(51) Int. Cl. A61L 2/00; A61F 7/12

(52) U.S. Cl. CPC A61L 2/0023; A61F 7/12; A61F 2007/0005

(57) ABSTRACT

Systems, devices and methods for thermal bacterial decolonization are provided. More particularly, in several embodiments, a heated fluid such as air or a liquid is directed to a body cavity or surface of a patient colonized or infected with bacteria in order to thermally decolonize that cavity or surface. In several embodiments, the systems, devices and methods disclosed herein achieve bacterial decolonization without risk of development of antibacterial resistance.

### BACKGROUND

Staphylococcus aureus is one of the most important pathogens in human disease. Infections with this microorganism create enormous direct and indirect costs due to the associated morbidity and mortality. Patients with S. aureus infections have longer hospital stays, larger medical expenses and can have 3-5 times the risk of in-hospital death. Treatment of S. aureus infections is a large concern in the medical community.

### SUMMARY

Treatment of bacterial infections with antibiotic therapies presents a number of disadvantages, chief among them the possibility of the bacteria developing resistance to the antibiotic. Thus, there is provided, in several embodiments, a system for the thermal decolonization of bacteria from a body cavity of a subject (such as a patient), the system comprising an elongate treatment member comprising a proximal end region in fluid communication with a source of decolonization fluid, and a distal end region configured to deliver a therapeutic amount of decolonization fluid (e.g., an amount of sufficient volume and at a sufficient temperature to inhibit, partially or completely, bacteria within or on the body cavity) to the body cavity, the treatment member comprising a fluid conduit extending between the proximal and distal end regions; and a heating unit operably coupled to one or both of the source of decolonization fluid and the treatment member, such that decolonization fluid can be heated prior to delivering the therapeutic amount of decolonization fluid to the body cavity. The decolonization fluid may be, for example, a liquid or a gas.

There is also provided a system for the thermal decolonization of bacteria from an anterior portion of an intranasal cavity of a subject, an example system comprising: an elongate treatment member comprising a proximal end region in fluid communication with a source of decolonization fluid, and a distal end region configured to deliver a therapeutic amount of decolonization fluid to the anterior portion of the intranasal cavity, the treatment member comprising a fluid conduit extending between the proximal and distal end regions; and a heating unit operably coupled to one or both of the source of decolonization fluid and the treatment member, such that decolonization fluid can be heated prior to delivering the therapeutic amount of decolonization fluid to the anterior portion of the intranasal cavity.

Provided herein are also methods for bacterial decolonization of a body cavity of a subject, comprising contacting a body cavity with heated decolonization fluid. In some embodiments, the decolonization fluid that contacts the bacteria in or on the body cavity of a subject is at a temperature of between about 110 and about 130 degrees Fahrenheit, including between about 110 and about 115 degrees Fahrenheit, about 115 to about 120 degrees Fahrenheit, about 120 to about 125 degrees Fahrenheit, about 125 to about 130 degrees Fahrenheit, and overlapping ranges thereof. In additional embodiments, substantially greater temperatures are optionally employed, for example from about 130 degrees Fahrenheit to about 220 degrees Fahrenheit, including about 130 degrees Fahrenheit to about 150 degrees Fahrenheit, about 150 degrees Fahrenheit to about 170 degrees Fahrenheit, about 170 degrees Fahrenheit to about 190 degrees Fahrenheit, about 190 degrees Fahrenheit to about 200 degrees Fahrenheit, about 200 degrees Fahrenheit to about 212 degrees Fahrenheit, about 212 degrees Fahrenheit to about 220 degrees Fahrenheit, and overlapping ranges thereof. In such embodiments wherein an elevated temperature is employed, treatment times are proportionately reduced, so as to avoid inducing thermal damage to normal tissue (or pain responses in the subject).

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a cross section schematic of the nasal cavities of a human.

FIG. 2 is a schematic diagram of one embodiment of the systems for thermal decolonization of the nasal cavity as disclosed herein.

FIG. 3 is a schematic diagram of an assembly configured to contain and dispense decolonization media and receive used decolonization media.

FIG. 4 is a cross sectional view of one embodiment of a heating device configured to heat decolonization media.

FIG. 5 is a cross sectional view of an additional embodiment of a heating device configured to heat decolonization media.

FIG. 6 is a cross sectional view of one embodiment of an occluding member configured to be positioned within a body cavity that is subject to decolonization.

FIG. 7 is a cross sectional view of one embodiment of the valve structure and decolonization media flow pathways of the occluding member.

FIGS. 8A-8C schematically depict expansion of the occluding member, according to several embodiments disclosed herein.

FIG. 9A is a schematic illustration of one embodiment of a device for delivery of decolonization media.

FIG. 9B is a schematic illustration of the proximal portion of one embodiment of a decolonization media delivery device.

FIG. 9C is a schematic illustration of the mid-portion of one embodiment of a decolonization media delivery device.

FIG. 9D is a schematic illustration of the distal portion of one embodiment of a decolonization media delivery device.

FIG. 10 is a schematic illustration providing a more detailed view of the distal portion of one embodiment of a decolonization media delivery device.

FIGS. 11A-11C depict various views of a valve system for use in several embodiments of a decolonization media delivery device.

FIG. 12 is a schematic illustration of one embodiment of decolonization media delivery device.

FIGS. 13A-13B depict decolonization media flow pathways as a result of various valve configurations within a decolonization media delivery device.

FIG. 14 schematically depicts one configuration of decolonization media flow pathways in the distal probe of one embodiment of a decolonization media delivery device.

FIG. 15 schematically depicts one variation of decolonization media flow pathways in the distal probe of one embodiment of a decolonization media delivery device.

FIG. 16 is a front oblique view of a distal probe depicting various decolonization media flow ports.

FIG. 17 is a rear oblique view of a distal probe depicting various decolonization media flow pathways.

FIGS. 18A-18B depict heat mapping of decolonization media emitted various embodiments of a distal probe having various decolonization media flow pathways.

FIG. 19 is a schematic illustration of an additional embodiment of decolonization media delivery device.

FIGS. 20A-20B depict cross-sectional schematics of the human ear and related anatomy.

FIGS. 21A-21B depict schematics of human ear canal.

FIG. 22 depicts a schematic of one embodiment of a system for thermal decolonization of the ear.

FIG. 23 depicts one embodiment of a pressure-sensitive pathway for deployment of decolonization media.

FIG. 24 depicts a cross-sectional schematic of one embodiment of a decolonization media delivery tube.

FIG. 25 depicts a cross-sectional schematic of an alternative embodiment system for thermal decolonization.

FIG. 26 is a schematic illustration of an additional embodiment of a decolonization media delivery device.

FIG. 27 is a schematic of a charging valve assembly in accordance with one embodiments of a decolonization media delivery device employing compressed gas to deliver the decolonization media.

FIG. 28 is a schematic of a plurality of apertures configured to adjust to maintain delivery rate of decolonization media present in the mid-distal region of one embodiment of a decolonization media delivery device.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Staphylococcus aureus is one of the most important pathogens in human disease. Approximately 2,000,000 hospital-acquired infections per year in the United States alone result in $30.5 billion in direct costs and significantly more in indirect costs. Staph infections cause 12,000 deaths per year in the U.S., with a direct cost of $9.5 billion. Patients with S. aureus infections have longer hospital stays, larger medical expenses and can have 3-5 times the risk of in-hospital death. The conundrum faced by many hospitals is the cost of implementing infection control measures, which are viewed as too great, yet hospitals rarely recoup the full costs of providing care to a patient who is hospitalized for weeks or months with infection.

Staph infections can result from staphylococci carried in and/or on patients and staff in hospitals and nursing homes, as well as people in the general community. Non-medical care providers may simply acquire staph from healthcare workers who are family members. In some cases casual contact or co-localization (e.g., sitting in a restaurant booth recently occupied by an infected hospital worker) are sufficient for microbial transfer.

A common site of staph colonization is the anterior nares (nostrils), which are especially likely to harbor hospital-acquired methicillin-resistant S. aureus (HA-MRSA). 80% of people carry staph nasally; 20% are persistent carriers, while 60% are intermittent carriers. Interestingly, S. aureus nasal carriers have increased risk of developing S. aureus infections at surgical sites or when outfitted with an intravenous catheter. Carriage rates are significantly greater in certain patient groups, such as diabetics and those on continuous ambulatory peritoneal dialysis. This may be due to the more frequent hospital visits of such patients. In patients with S. aureus bacteremia (the presence of bacteria in the blood), blood isolates have been shown to be identical to those from the anterior nares in over 80% of infected patients. This work suggests that many cases of S. aureus bacteremia originate from nasal colonization. Thus, as accomplished by several embodiments disclosed herein, reduction or elimination of nasal S. aureus carriage reduces infection rates.

Staph nasal carriage is typically treated by nasal ointments or oral antibiotics, but the efficacy of such treatments is variable and raises the risk of generation of antibiotic resistant strains. Furthermore, oral antibiotics are often expensive and trigger side effects that limit patient compliance, which heightens the risk of antibiotic resistance. Topical (nasal) antibiotics can be messy and may require repeated applications that contribute to poor patient compliance and the emergence of antibiotic resistance. Mupirocin (BACTROBAN OINTMENT®), used by some physicians as a topical nasal antibiotic for treating staph nasal carriage, has been associated with some treatment failures and induction of bacterial resistance. This may be due to muciprocin being highly protein bound (>97%), which could reduce its effectiveness in the presence of proteinaceous secretions, which are present in the nose. Moreover, the effect of such nasal secretions on the concentrations of mupirocin needed to inhibit S. aureus growth have not be formally established. The lack of such data is important because the minimum concentrations required to achieve a bacteriocidal effect (MBC) against relevant intranasal pathogens can range from 8-fold to 30-fold higher than the minimum inhibitory concentration (MIC).

Another common location of S. aureus colonization is the external auditory canals (e.g., ear canals or external acoustic meatus). Topical antibiotics are commonly used to treat colonization or infection, but often fail because of antibiotic resistance or poor patient compliance. Otic drops can be messy as a consequence of their low viscosity, which permits gravity to pull the drops downward, onto the inferior (bottom) surface of the ear canal, and then onto the pinna (auricle or external ear). This messiness tends to discourage frequent use, thereby heightening the risk of treatment failure and emergence of bacterial resistance.

Successful treatment of auditory infections, if ever achieved, can take weeks, during which time patients may be bothered by the frequent antibiotic instillations and auricular wetness. Resolution of colonization or infection can be further delayed or entirely prevented by cerumen (earwax), which often blocks direct contact of the antibiotic with the germs. Physicians have long presumed that wet cerumen (as opposed to dry, flaky cerumen) exerts a bactericidal effect, but it may actually encourage bacterial growth. Rapid resolution of infection is desirable for all patients but critical for patients with compromised immunity, such as those with HIV or diabetes, who are at increased risk for potentially life-threatening complications.

S. aureus (including methicillin resistant S. aureus (MRSA)) colonization of the ear canals is a particular problem in hospitals, clinics, and nursing homes, where personnel frequently spread the microorganisms to patients, causing infection and often death. Habitual contacting of the hands and/or fingers with ear canal openings or frequent contact with stethoscope earpieces allows transmission of resident bacteria to a subject's hands and/or a stethoscope, which may lead, directly or indirectly, to distribution of S. aureus to patients. These infections and their transmission present a common and serious problem. “Infections picked up in hospital affect almost a third of patients in intensive care, and kill 44% of those people” (see Terry Tudor. Overcoming attitudes and perceptions towards the management of infections and waste in the hospital setting: a case study from the UK. International Journal of Behavioural and Healthcare Research, 2011; 2 (4): 307-319)).

The increasing resistance of S. aureus to common antibiotics and the prevalence of infections (and costs associated therewith) indicates that effective measures, such as those devices and methods disclosed herein, to prevent S. aureus infections are urgently needed.

This description relates generally to devices and methods used to heat a portion of the body, such as an epithelial structure (e.g., the nasal or otic cavities), at least in part, to a temperature sufficient to decolonize the structure, or portion thereof, of pathogens, such as bacteria, such as, for example, S. aureus. In several embodiments, the decolonization is achieved by contact of the colonized surface with a decolonization media that is of the temperatures disclosed herein. In several embodiments, the decolonization media temperatures achieved by the disclosed devices and methods range from about 115° F. to about 130° F., which are bacteriocidal in several embodiments (e.g., the temperature of the media when it contacts the pathogen, thereby resulting in reduced viability of the pathogen). In several embodiments, the temperatures range from 115° F. to about 117° F., 117° F. to about 119° F., 119° F. to about 121° F., 121° F. to about 123° F., 125° F. to about 127° F., 127° F. to about 129° F., and overlapping ranges thereof. In several embodiments, the bacteriocidal temperatures achieved are between about 120° F. to about 122° F. In some embodiments, the temperature of the decolonization fluid is selected as a temperature sufficient to decolonize the body cavity of at least one pathogen that may be present in the body cavity. In some embodiments, the temperature of the decolonization temperature is selected as the highest temperature that does not induce unacceptable pain in the subject, for example a temperature at or proximate to the pain threshold for the subject, or a typical subject. In some embodiments, the temperature of a decolonization fluid may be a time-averaged temperature, and the instant temperature may vary as a function of time, allowing higher temperatures to be transiently applied for short time periods without triggering a severe pain response. In some embodiments, the temperature may be a spatially averaged temperature, and local variations may be introduced e.g. through co-propagating fluid streams of differing temperature. Additionally, anesthesia is used in some embodiments, to reduce risk of pain response during or after treatment. In several embodiments, temperatures above 122° F., even substantially above, are used. For example, in several embodiments temperatures of the decolonization media may range from about 120° F. to about 140° F., about 140° F. to about 160° F., about 160° F. to about 180° F., about 180° F. to about 200° F., about 200° F. to about 220° F., or any temperature in between the listed ranges. Depending on the decolonization media (e.g., fluid or gas), greater temperatures may also be used. In several such embodiments employing elevated temperatures, the tissue damage is reduced and/or avoided by one or more of pre-cooling the treatment area, limiting the thermal application time, and controlling the density and droplet size of the decolonization media (the latter in the case of liquid media). With such pre-treatments (or concurrent treatments) tissues can safely contact decolonization media heated substantially above what is commonly believed to result in thermal injury. In some embodiments, minor thermal injury to tissue potentiates the decolonization effects (e.g., by induction of localized inflammation and/or immune responses).

In several embodiments, the decolonization is achieved by contact (e.g., thermal heating) of the colonized surface with a decolonization medium. In several embodiments, the decolonization medium comprises air (or another gas), which is optionally humidified. In several embodiments, a vacuum feature of the devices disclosed herein (discussed in more detail below) functions to limit and/or avoid heat plume propagation. In several embodiments, the decolonization medium comprises a fluid (or one or more fluids). In several embodiments, the temperature of the decolonization medium within the device (or as it travels along the device) is greater than the temperature of the medium when it contacts the pathogens (e.g., the devices can be configured to account for temperature loss, if needed). Decolonization may include removal of some or all of each of one or more pathogen species from a body cavity or a portion thereof.

In particular, in several embodiments there is provided a system for the thermal decolonization of bacteria from a body cavity of a subject, that comprises a treatment member comprising a fluid conduit and in fluid communication with a source of decolonization fluid and configured to deliver a therapeutic amount of decolonization fluid to the body cavity, and a heating unit operably coupled to one or both of the source of decolonization fluid and the treatment member, such that decolonization fluid can be heated prior to delivering the therapeutic amount of decolonization fluid to the body cavity. The treatment member may be elongate. For example, the treatment member may have a length to width ratio of at least 2.

In several embodiments, the heating unit is configured to heat the decolonization fluid to a temperature of between about 110 to about 125 degrees Fahrenheit (including about 110 to about 115 degrees, about 115 to about 120 degrees, about 120 to about 125 degrees, about 119 to about 122 degrees, and overlapping ranges thereof). In some embodiments, the heating unit is configured to maintain the decolonization fluid at a constant temperature of about 120 degrees Fahrenheit. Depending on the embodiment, the heating element heats the decolonization fluid by one or more of direct heating, inductive heating, infrared heating, halogen heating, and/or microwave heating. In several embodiments, the heating unit is configured to heat the decolonization fluid to higher temperature range, for example, between about 120 to about 150 degrees Fahrenheit (including about 120 to about 125 degrees, about 125 to about 135 degrees, about 135 to about 145 degrees, about 145 to about 150 degrees, and overlapping ranges thereof). In several such embodiments, the increased temperature allows decolonization of topical surfaces that are less temperature sensitive.

In several embodiments, the system further comprises a fluid receptacle and the elongate treatment member further comprises a second fluid conduit for carrying decolonization fluid away from the body cavity to the fluid receptacle. In some embodiments, the fluid receptacle is integral with the treatment member, while in some embodiments, it is a separate element.

In several embodiments, the elongate treatment member further comprises a temperature sensor, such as a thermistor or thermocouple, configured to detect the temperature of the decolonization fluid (e.g., placed in or along the fluid conduit), and or the temperature in the body cavity. The temperature sensor may be in electrical communication with the heating unit, and provide a temperature signal to the heating unit. The heating unit may include an electronic heating control circuit that receives the temperature signal and regulates the heating of the decolonization fluid in a manner than ensures decolonization, but minimizes heat buildup in the target tissue.

In some embodiments, temperature control systems are employed to maintain the temperature of the decolonization media in a narrower temperature range, thereby reducing temperature buildup in the target tissue. In several embodiments, the elongate treatment member (or another portion of the device, comprises a sensor, such as a thermistor or thermocouple, configured to detect the temperature of the decolonization fluid passing through a non-heated (and/or non-insulated) passage prior to emission. Such embodiments can compensate for temperature loss/gain due to the ambient environment. Moreover, in several such embodiments, a humidity sensor is additionally included to account for temperature fluctuations due to humidity. The temperature and/or humidity sensors may be in electrical communication with the heating unit, and provide a temperature signal to the heating unit, such that the temperature of the fluid can be adjusted up or down to account for loss or gain.

In several embodiments, a narrower temperature range is achieved by sensing the initial temperature of a target tissue (or the ambient temperature of a target cavity) prior to emission of decolonization media from the treatment member. As above, the temperature can be sensed by using a thermistor or thermocouple, either alone or in combination with a humidity sensor. Additionally, the target tissue can be exposed to a sample heating period in order to determine the approximate amount of buildup that will occur in that particular tissue. With those pieces of information, the treatment system can be configured to increase or decrease the temperature of the decolonization media to be emitted from the elongate treatment member.

Additionally, fine control of temperature can be achieved by pre-heating emission pathways of the treatment member prior to actually beginning a decolonization treatment. For example, heated decolonization media (or other heated fluid or gas) can be passed through the emission pathways of the treatment member during a warm-up phase. This warm-up phase functions, in some embodiments, to warm at least the inner surfaces of the emission pathways such that there is a reduction in the amount of heat loss from the decolonization media during a decolonization treatment. In essence, the difference in temperature between the decolonization media and of some of the emission pathways is reduced, thereby minimizing heat loss and imparting a finer control of decolonization media temperature. In some embodiments this is achieved by a distal temperature sensor (either integrated into or separate from the treatment member) that monitors the temperature of the decolonization media exiting device and returns a signal to the heating unit that can increase or decrease the temperature of the decolonization media according to the amount of temperature loss (or lack thereof) that occurs as the decolonization media passes through the emission pathways. In additional embodiments, the treatment member may comprise specifically designed emission pathways that function, in essence, as heatsinks. In such embodiments, the increased thermal mass of surfaces that come into contact with heated decolonization media reduces the temperature loss of the decolonization media as it passes over such surfaces.

In additional embodiments, narrow temperature control ranges of decolonization media are achieved through the use of proportional-integral-derivative (PID) controllers, which calculate an “error” value between a desired setpoint (e.g., the target temperature of decolonization media as it exits the treatment member) and the actual value of the variable of interest (e.g., the actual temperature of the media). Using “gain scheduling” the desired target temperature of the decolonization media can be fed-forward into a temperature controller, the actual temperature measured by a temperature sensor and the difference between them (e.g., the “error”) compensated for by heating or cooling the decolonization fluid. In several embodiments, cascading (or nested) PID controllers are used to further fine tune the temperature control.

In several embodiments, the system further comprises an outer sealing member positionable outside the body cavity, the outer sealing member being configured to retain heated decolonization fluid in the body cavity and prevent unintended drainage of decolonization fluid from the body cavity. In several embodiments, the system further comprises an inner sealing member positionable within the body cavity that is configured to retain heated decolonization fluid in the body cavity and prevent unintended migration of decolonization fluid from the body cavity to an adjacent portion of the body. In several embodiments, one or more of the sealing members is an inflatable balloon. In one embodiment, the inner sealing member is an inflatable balloon. In one embodiment, the outer sealing member comprises a nostril cup.

In several embodiments, the decolonization fluid comprises an aqueous medium, such as water or saline solution. In several embodiments, the decolonization fluid comprises a gas, such as air, nitrogen, or one or more other gases. In several embodiments, the source of decolonization fluid comprises one or more reservoirs. Depending on the embodiment, the one or more reservoirs operate by gravity while in other embodiments the reservoirs are pump operated. Combinations of pumps, gravity, and/or vacuum are used in additional embodiments.

In several embodiments, the system is configured for the thermal decolonization of the intranasal cavity of a subject. In several embodiments the anterior portion of the intranasal cavity of the subject is decolonized. In several embodiments, the system further comprises a occluding member configured to reversibly seal at least one nostril of the subject, thereby causing heated decolonization fluid to remain in the anterior portion of the intranasal cavity, and further comprises at least one nostril cup operably connected to a second fluid conduit in the elongate treatment member, the second fluid conduit being configured to convey decolonization fluid away from the anterior portion of the intranasal cavity. In one embodiment, the occluding member comprises a reversibly inflatable balloon.

In several embodiments, the nostril cup comprises an axial lumen having an inner diameter that engages the outer diameter of the second fluid conduit. In several embodiments, the nostril cup comprises a plurality of valves that allow fluid communication between the inner portion of the nostril cup and the anterior portion of the intranasal cavity when the valves are in an open position and maintain the decolonization fluid in the anterior portion of the intranasal cavity when the valves are in a closed position. In several embodiments, the valves are pressure-sensitive, while in alternative embodiments, the valves are electronic or otherwise controlled.

In one embodiment the valves are flap valves that open in response to pressure that exceeds a threshold, thereby allowing used decolonization fluid to flow into the inner portion of the nostril cup and into the second fluid conduit.

Certain embodiments also comprise a fluid receptacle coupled to the second fluid conduit, the fluid receptacle being configured to receive used decolonization fluid.

In several embodiments, the source of decolonization fluid comprises a reservoir. In several embodiments, the reservoir comprises a mixing device configured to facilitate heating of decolonization fluid within the reservoir by convection. Depending on the embodiment, the mixing device comprises one or more of a shaker, a rocker, a magnetic stir plate, a rotatable or translatable stirring element, a roller and combinations thereof. In several embodiments, the mixing device comprises a rocker comprising opposing plates between which the reservoir is positioned, with at least one of the opposing plates is configured to heat the decolonization fluid within the reservoir. In several embodiments, the system also comprises a sensor for detecting a temperature of the decolonization fluid within the reservoir. In one embodiment, at least one of the opposing plates is flexible and suitable for conforming to a surface of the reservoir. In one embodiment using a rocker as a mixing device, the rocker is rocked about an axis by a motor.

In several embodiments, the opposing plates are coupled to a means for urging the plates towards one another, such that the reservoir may be pressurized, thereby facilitating transmission of the decolonization fluid to the anterior intranasal cavity of the subject. In additional embodiments, the system further comprises a pneumatic sleeve configured to surround at least a portion of the reservoir, the pneumatic sleeve being coupled to a pneumatic pressure source, such that the reservoir may be pressurized by actuation of the pneumatic pressure source, thereby facilitating transmission of the decolonization fluid to the anterior portion of the intranasal cavity of the subject.

In several embodiments, the system further comprises a connector that fluidly couples the fluid receptacle and the reservoir. Depending on the embodiments, the connector, the fluid receptacle and the reservoir are disposable or are reusable. In several embodiments, the connector, the fluid receptacle and the reservoir are unity (e.g., manufactured or connected as a single unit) while in other embodiments one or more of the connector, the fluid receptacle and the reservoir are separate.

In several embodiments, the reservoir is variably positionable above the intranasal cavity of the subject such that gravity-induced pressure is sufficient to deliver decolonization fluid to the anterior portion of the intranasal cavity. Such embodiments, optionally comprise an indicator for positioning in line with the subject's line of sight, such that visualization of the indicator by the subject indicates a placement of the reservoir in a suitable position for gravity-induced delivery of decolonization fluid.

In additional embodiments, the systems are configured for the thermal decolonization of the otic cavity (e.g., the inner ear canal). In such embodiments, the system comprises a sealing portion comprising a chamber through which proximal to distal and a distal to proximal fluid conduits pass, the chamber comprising a distally positioned conformable gasket configured to seal around the external portion of the ear canal, and a proximally positioned fail-safe vent configured to release decolonization fluid that exceeds the flow capacity of the fluid conduits. In one embodiment, the system is also suitable for removal of ear wax or pus.

In several embodiments, the system comprises at least two reservoirs housing decolonization fluid; a first reservoir houses decolonization fluid that is heated, and a second reservoir houses decolonization fluid that is cool (at least with respect to the temperature of the heated fluid). In several embodiments, the first reservoir housing heated decolonization fluid and the second reservoir housing cool decolonization fluid are coupled to a valve in fluid communication with the proximal to distal fluid flow pathway.

In several embodiments, the system further comprises a microcontroller that receives information regarding the average temperature of the heated and cool decolonization fluids and controls the valve to regulate the temperature of the decolonization fluid. In some embodiments, an electronic control circuit, for example including a microprocessor, may be used for one or more functions, such as control of a heating element, control of a cooling element (such as a fan, thermoelectric cooling element, or source of cool fluid), fluid pressure control, humidity of a gaseous decolonization fluid, timing of decolonization fluid administration, provision of an alert if a monitored temperature falls outside of a predetermined temperature range, and provision of entertainment to the patient during administration of decolonization fluid.

In some embodiments, the proximal to distal fluid flow pathway and the distal to proximal fluid flow pathway each further comprise a pump to instill or remove the decolonization media from the inner ear canal (or other body cavity) of the subject.

In several embodiments, the proximal to distal fluid conduit comprises a pressure sensor at its distal portion. In several embodiments, the pressure sensor functions to limit the peak pressure of decolonization fluid within the inner ear canal and/or to sense the fluid level within the sealing portion positioned outside the body cavity. In one embodiment, the fluid conduit also comprises a temperature sensor (such as a thermistor or other temperature sensitive device) within its distal portion to sense the temperature of the delivered decolonization media. In several embodiments, the distal portion of the proximal to distal fluid flow pathway comprises a plurality of orifices that have increasing diameter from a proximal to distal direction along the length of the pathway.

In several embodiments, operation of the system results in heated and cool decolonization fluid being applied sequentially in order to remove approximately as much heat as was transferred to the ear canal of the subject.

In several embodiments, for efficiency and precision in operation of the system, one or more components of the system are insulated to reduce heat exchange with the environment. In several embodiments, the insulation comprises one or more insulating materials selected from the group consisting of metal foil, foam (such as closed-cell foam), aerogel, silica-aerogel, and silica-aerogel with an opacifier agent. Other example insulating layers materials include other porous materials, and materials including insulating layers such as air-filled layers.

In several embodiments, the source of decolonization fluid is the environment external to the elongate treatment member. In several embodiments, an elongate treatment member further optionally comprises a motor for conveying the decolonization fluid along the fluid conduit and to the body cavity. In several embodiments, the system also comprises a motor for retrieving the used decolonization fluid from the body cavity and conveying the used decolonization fluid through the fluid conduit and back to the environment, for example to a receptacle such as a storage tank.

Optionally, several embodiments of the system using air for decolonization further comprise a water tank and a humidifier to humidify the air. Additionally, such embodiments, may optionally further comprise a hygrometer to detect the relative humidity of the humidified air.

In several embodiments, the distal portion (e.g., the treatment portion) of the system comprises a probe located at the distal end region of the elongate member and configured for insertion into the body cavity, the probe having at least one apical port and at least one lateral port, the apical port(s) and lateral port(s) being configured to expel or retrieve decolonization fluid from the body cavity. In several embodiments, there are a plurality of valves configured to alternate expulsion of heated decolonization fluid from the apical port(s) and lateral port(s). In several embodiments, the probe is configured to expel the decolonization fluid from the at least one apical port and retrieve the used decolonization fluid from the body cavity via the at least one lateral port. In another embodiment, the probe is configured to expel the decolonization fluid from the at least one lateral port and retrieve the used decolonization fluid from the body cavity via the at least one apical port.

In several embodiments, the systems disclosed herein are suitable for the decolonization of thermal decolonization of a body cavity colonized by antibiotic resistant bacteria. In several embodiments, the antibiotic resistant bacteria comprise methicillin-resistant Staphylococcus aureus.

In several embodiments, there are also provided methods for bacterial decolonization of a body cavity of a subject, the methods comprising contacting a body cavity with heated decolonization fluid that is heated to a temperature of between about 110 and about 125 degrees Fahrenheit. Depending on the embodiments, the body cavity is selected from the group consisting of the anterior portion of the nasal cavity and the inner ear canal.

In several embodiments, the contacting is performed by inserting into the body cavity of the subject an elongate treatment member configured for transmission of heated decolonization fluid to a surface of the body cavity. Additionally, the methods optionally further comprise positioning a sealing member adjacent the opening of the body cavity in order to prevent unintended loss of decolonization fluid from the body cavity. In several embodiments, the methods also comprise positioning a sealing member positioned within the body cavity to prevent unintended translocation of the decolonization fluid from the body cavity to an adjacent portion of the body. In certain embodiments, the body cavity is the anterior portion of the nasal cavity and the sealing member prevents translocation of the decolonization fluid to the posterior portion of the nasal cavity.

In several embodiments, the methods are directed to the decolonization of non-endogenous bacteria. In several embodiments, the bacteria are resistant to antibiotics. In several embodiments, the antibiotic resistant bacteria comprise methicillin-resistant Staphylococcus aureus.

### Nasal Thermal Decolonization

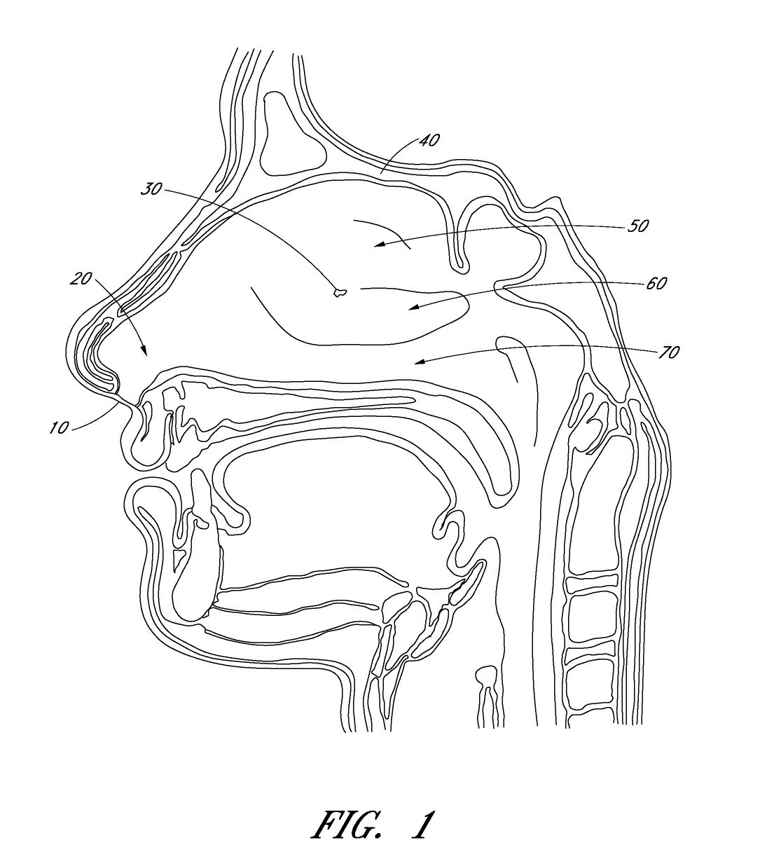

As discussed above, the nasal cavity is a frequent site of bacterial colonization. FIG. 1 depicts the relevant nasal anatomy. Bacteria access the nasal cavity nostrils 10 which leads to the nasal vestibule 20. The nasal vestibule bears small hairs called vibrissae that filter airborne particulate matter. The nasal vestibule is lined with keratinized stratified squamous epithelium; more posterior areas of the nasal cavity are lined with transitional and respiratory epithelia. The nasal turbinates (or conchae) are curled bony shelves located laterally in the nasal cavities and function to increase nasal surface area (for climate control: humidification, heating, and filtering). The middle turbinates 60 lie above the inferior turbinates 70 and act as buffers to protect the sinuses from coming in direct contact with pressurized nasal airflow, while the superior turbinates 50 serve to protect the olfactory bulb. The turbinates surround the opening to the maxillary sinus 30 and lie just inferior to the olfactory epithelium 40.

As discussed above, temperatures between about 110° F. to about 150° F. (or other temperatures, depending on the embodiment, such as, for example, between about 110° F. to about 115° F., about 115° F. to about 120° F., about 120° F. to about 122° F., about 122° F. to about 125° F., about 125° F. to about 130° F., about 130° F. to about 140° F., about 140° F. to about 145° F., about 145° F. to about 150° F., and overlapping ranges thereof) are used in several embodiments to decolonize the nasal epithelium staph by directing a heated decolonization medium (in several embodiments, the medium comprises an aqueous medium such as saline) into the nasal cavity. In several embodiments, as discussed more below, nasal mapping is used verify locations to which the staph colonization is confined. In several such embodiments, an imaging device (e.g., a sensor, location recorder/transmitter, camera, etc.) may be included in the device to facilitate mapping. In several embodiments colonization is primarily, substantially, or completely limited to the anterior portion of the nose (e.g., the vestibule and anterior naris). The vestibule and anterior naris are lined with keratinized stratified squamous epithelium, or KSSE, which can become colonized. Also, the nonciliated transitional epithelium that spans between the KSSE and the ciliated respiratory epithelium that lines most of the posterior nasal cavity may also be affected in some cases. These different cell types, and the ecological niches they inhabit, affect their susceptibility to staph colonization. In several embodiments, the conveniently accessed anterior nose is the only tissue that is treated while successfully achieving staph nasal decolonization. The posterior nasal cavity contains sensitive structures (such as the ciliated respiratory epithelium and the olfactory epithelium).

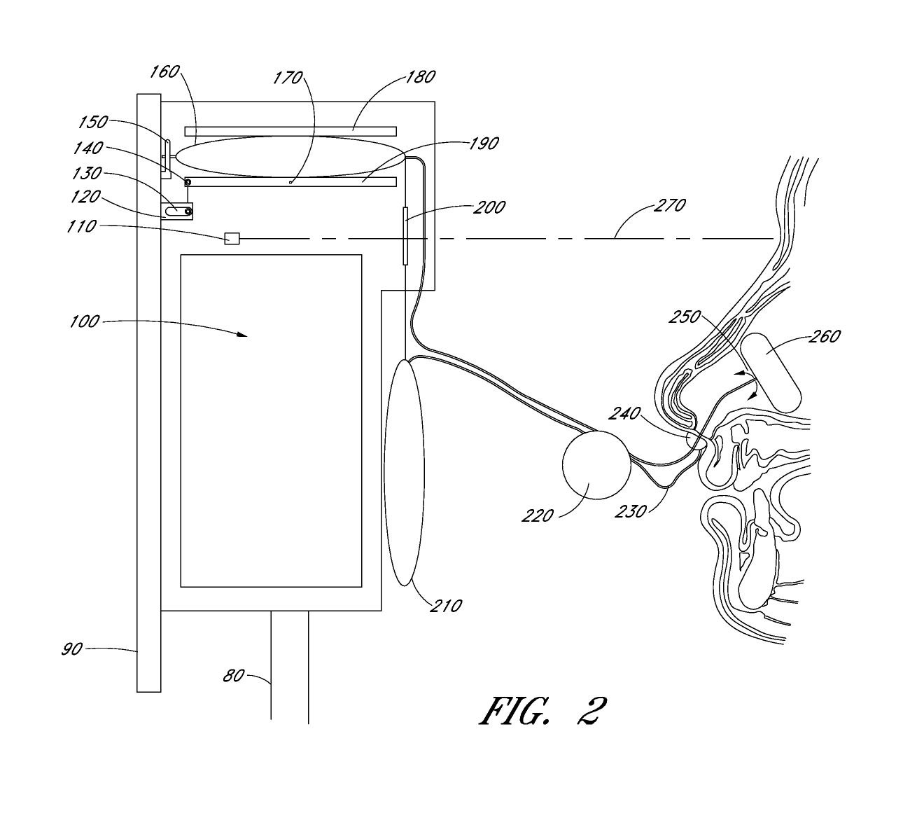

FIG. 2 depicts one embodiment of an administration system used for nasal decolonization. The system, in several embodiments, comprises an interface 100 that is mounted on a slideable pole 80 and/or slideable wall mount 90. Additional components of the system are discussed in greater detail below.

In several embodiments, the system comprises a multi-compartment container with three main portions, an upper media reservoir 160, a connector portion 200, and a lower media reservoir 210 (see also FIG. 3). In several embodiments, the multi-compartment chamber is single-use and disposable. In other embodiments, it can be sterilized and reused. In several embodiments, the upper reservoir contains decolonization media (e.g., saline in some embodiments) and the lower media reservoir is initially empty. The multi-compartment container is fixed into administration system by sliding an attachment rod 150 through an attachment hole 280 in an attachment tab 290 (see, e.g., FIG. 3). In several embodiments, other attachment mechanisms are used (e.g., adhesive, hook and loop fasters, etc.).

The upper media reservoir 160 is in fluid communication with an ingress fluid conduit 225 that directs decolonization media from the reservoir 160 to the nostril(s) of the subject being treated. Annularly surrounding the ingress fluid conduit 225 and functioning to seal the outer portion of the nostril is a nostril cup 240. The decolonization media flows 250 into the nostril to thermally kill the bacteria colonizing the nostril. Because the majority of the colonization is limited to the anterior portion of the nostrils, in several embodiments, physical blockage of heated decolonization media is performed to separate the anterior cavity from the posterior nasal cavity (and thus maintain the heated decolonization media in the anterior cavity). In several embodiments, and schematically depicted in FIG. 2, a pneumatic sealing balloon 260 is used, though in several embodiments, alternative blocking mechanisms are used (e.g., self-expanding polymer, shape-memory materials, etc). For example, a resilient plug may be inserted into the nostril to be decolonized before treatment, to prevent the decolonization medium from flowing further back into the sinuses. The seal between the cavities makes it possible, in several embodiments to used heated liquid decolonization media as opposed to heated gas, such as heated air. In several embodiments, a liquid medium achieves more rapid decolonization than is achieved with heated air. However, in several embodiments, heated air is preferred, for example in those patients who are less tolerant to a temporary insertion of an inflatable sealing device. The pneumatic sealing balloon 260 is in communication with an inflation bulb 220, such that pressure on the inflation bulb causes expansion of the sealing balloon 260 and sealing off of the anterior nasal cavity from the posterior nasal cavity. Thus, when inflated the pneumatic sealing balloon blocks the flow 250 of decolonization media. As the decolonization media returns to the nostrils, it is captured by the nostril cup 240 and conducted down the egress fluid conduit 230 to eventually be housed (and disposed of) in the lower media reservoir 210.

FIG. 3 depicts one embodiment of a multi-compartment container comprising an attachment hole 280 passing through an attachment tab 290 that reversibly interacts with the attachment rod 150 of the administration system. In several embodiments the connecting portion comprising a fenestration 310 (FIG. 3) that enables proper alignment of the multi-compartment container in the administration system. Returning to FIG. 2, in several embodiments, the fenestration 310 allows the height of the system to be adjusted along the sliding mount 80 such that the subject can visualize an indicator 110 through the fenestration via a line of sight 270.

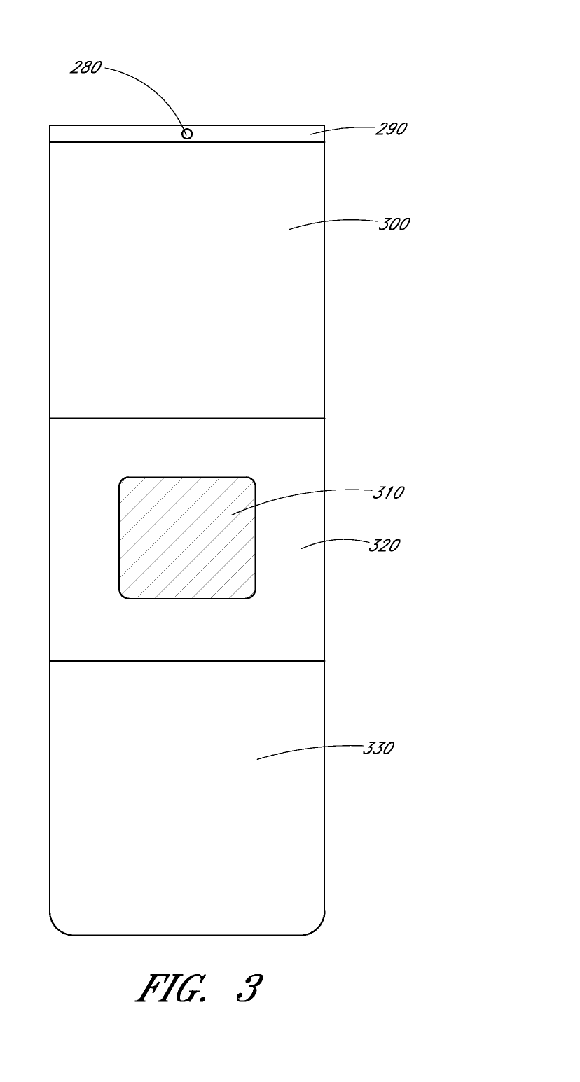

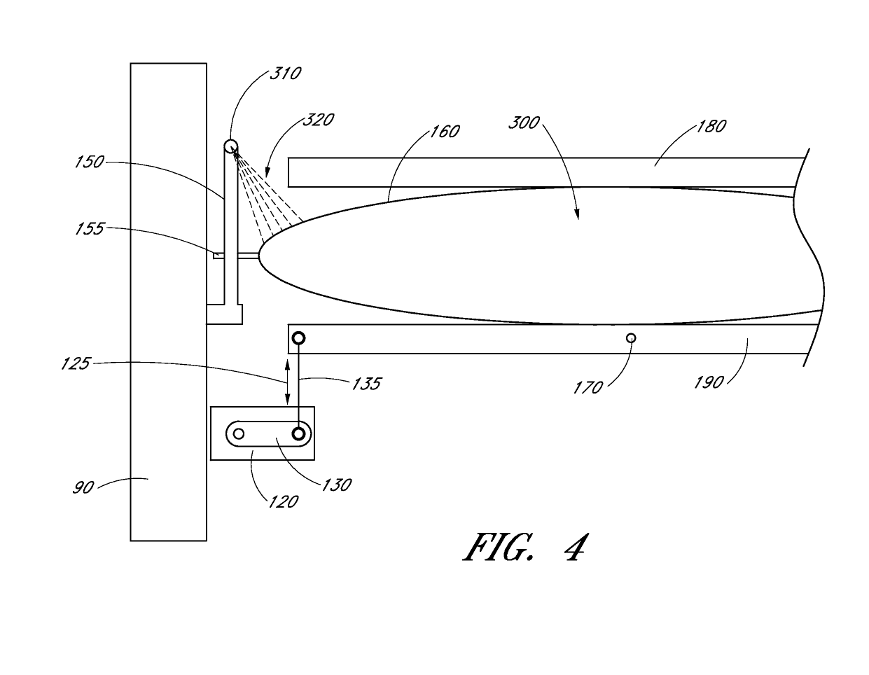

Certain aspects of an example system are shown in greater detail in FIGS. 4 and 5. In several embodiments, the upper reservoir 160 is sandwiched between an upper heater 180 and a lower rocking heater 190 that pivots about the rocking axis 170 when a servo (or servomechanism) 120 moves the servo arm 130 and servo connector 135 that couples it to the rocking heater 190. Motion of the servo connector 125 accelerates heating by inducing fluid convection. The upper heater, in several embodiments, is a rigid plate. However, if the upper heater is flexible or otherwise amenable to being shaped, it can be positioned to drape over the upper reservoir. In several embodiments, the contact enhances heat transfer to the decolonization media 300 and thus reduces heating time. In several embodiments, the weight of the upper heater also functions to place pressure on the decolonization media, such that flow into the nasal cavity is improved. In other embodiments, the upper heater is self-supported and does not substantially contact or directly contact the upper reservoir. In such embodiments, the decolonization media is driven into the nose by gravity alone (although pumps, vacuum or other mechanisms can also be used). In such embodiments, the upper heater can be configured to heat by a different mechanism (e.g., non-contact). Such non-contact methods of heating (or sensing heat) include, but are not limited to, infrared, halogen, microwave, and the like. As shown in FIG. 4, several embodiments comprise a temperature sensor (such as an infra-red sensor) 310 to detect the temperature of the decolonization medium 300 and adjust the heater plates 180 and 190 to precisely control the temperature of the decolonization media 300. In several embodiments, schematically depicted in FIG. 5, an “island” of material 330 that is a poor heat conductor (relative to the heating plate 180, such as for example, foam, rubber, etc.) surrounds a sensor 340 (such as for example a thermistor)), senses the temperature of the upper reservoir 160 (and hence the contained decolonization media 300). In several embodiments, to minimize false temperature readings the thermal island 330 is surrounded by a ring or layer of metal (e.g., aluminum, steel, etc.) that blocks infrared radiation from the heater plate 160.

Once the temperature sensor of a particular embodiment establishes that the temperature of the decolonization media 300 is at an appropriate temperature for decolonization (e.g., between about 115° F. to about 130° F., about 115° F. to about 120° F., about 120° F. to about 122° F., about 122° F. to about 125° F., about 125° F. to about 130° F., and overlapping ranges thereof in several embodiments) the interface 100 prompts a user (or does so automatically) to squeeze the inflation bulb 220, thereby inflating the pneumatic sealing balloon 260 and sealing the anterior nasal cavity from the posterior nasal cavity. While the pneumatic sealing balloon 260 is depicted schematically as a pancake shape, other shapes (e.g., round balloons) are used in other embodiments. In certain embodiments, a balloon that is customized to the nasal cavity of a subject is generated and used, to ensure adequate sealing.

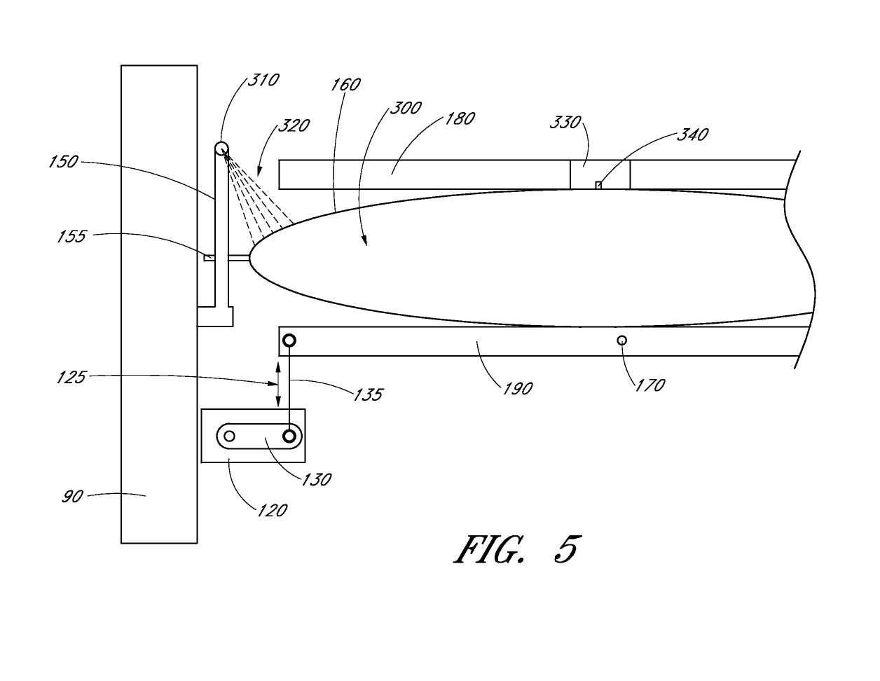

As discussed above, the flow 250 of heated decolonization media out of the ingress fluid conduit 225 decolonizes the anterior nasal cavity based on the elevated temperature of the media. The media is collected in the nostril cup 240, which as shown in FIG. 6 comprises an axial lumen 370 which frictionally engages with and slides over the ingress fluid conduit 225 (see movement 480 in FIG. 7). This permits the cup 240 to snugly seal the nostril so fluid leaves only via the egress fluid conduit 230 and tether the pneumatic sealing balloon 260 in place during treatment. In several embodiments, the egress fluid conduit 230 is separated from the ingress fluid conduit 225 at least for a portion (see e.g., FIG. 2) that eases the longitudinal motion of the nasal cup along the ingress fluid conduit 225. In other embodiments, the cup 240 is unitary (e.g., formed with or as a part of the ingress fluid conduit 225). In several embodiments, the cup 240 comprises an outer shell 380, which comprises a plurality of valves 360a, 360b, etc. which are free at their inner portions 390 but attached (e.g., hinged) to the cup at a hinge portion 400. When opened by flow of decolonization media from the nasal cavity, the valves allow the fluid to enter the egress fluid conduit 230 and flow to the lower media reservoir 210. While valves are used, in several embodiments, at least in part because they open only after a sufficient back pressure is reached, in other embodiments, drain holes (e.g., always open) are used. In some embodiments with valves, the valves are constructed such that they open only when the decolonization media delivered to the nostril is sufficient to fill all areas in the nostril anterior to the pneumatic sealing balloon 260. This aids in assuring the sufficient distribution and decolonization of all portions of the nostril anterior to the pneumatic sealing balloon 260. The valves also automatically regulate back pressure because, they close if pressure drops, thus retaining more fluid and hence increasing pressure; they open as pressure rises to the desired back pressure, they open more if pressure rises above the desired back pressure (thereby acting as a safety/comfort feature).

As shown in cross-section in FIG. 7, the nostril cup 240 has a convex outer shell 380 that defines an interior volume 440. The interior volume 440 receives decolonization media from the nostrils via a pathway 430 defined by open valves (360a). Closed valves 360b maintain decolonization media in the nostril until a threshold pressure is reached. Once in the inner volume 440, the decolonization media enters the egress fluid conduit 230. In several embodiments, the valves are closed in a resting position (e.g., no pressure gradient). In additional embodiments, a single valve is optionally used (e.g., in place of, for example, 360b). In such embodiments, the single valve is placed, for example at the most distal portion of the egress fluid conduit 230, at the point where it joins the outer shell 380.

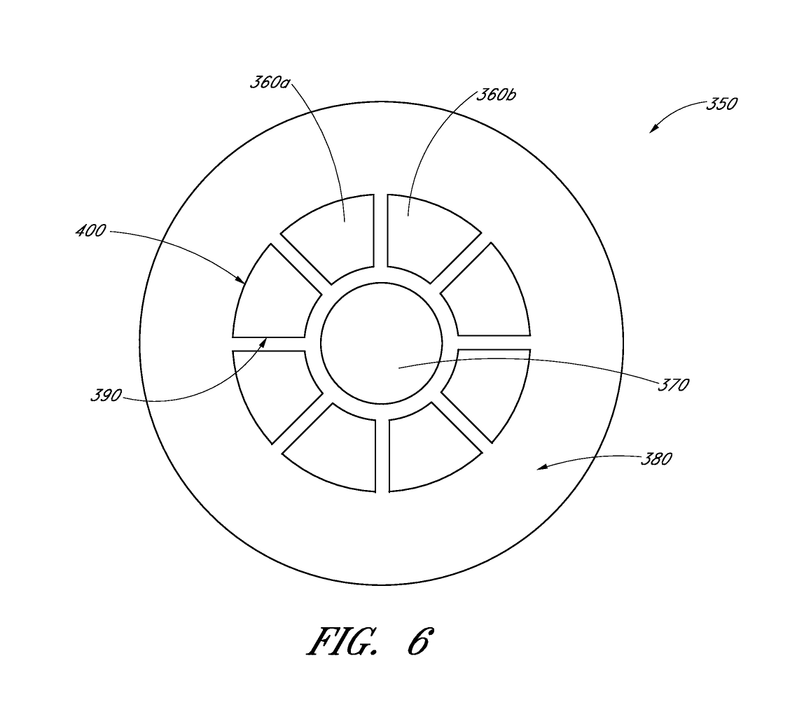

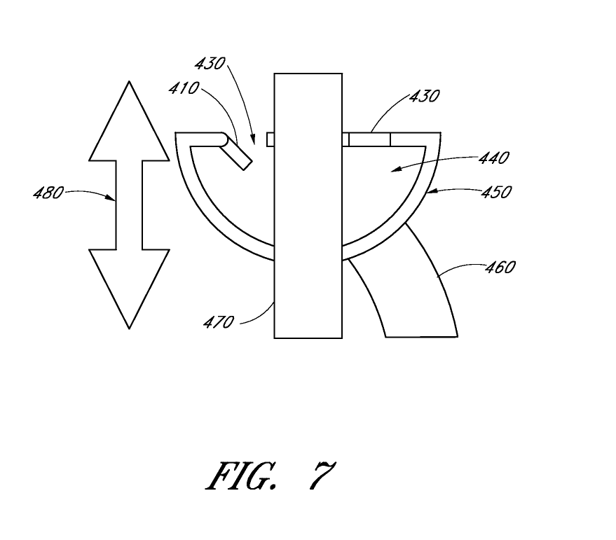

As shown schematically in FIGS. 8A-8C, the inflation bulb 220, in response from pressure applied thereto causes the inflation of the pneumatic sealing balloon 260 (see increased inflation 260a, 260b, and 260c in FIGS. 8A, 8B, and 8C, respectively). In several embodiments, the inflation bulb 220 is automated and, for example, comprises a small air compressor within the system. In such embodiments, the pneumatic sealing balloon 260 seals automatically and to a pressure within a certain predetermined range for adequate safety, comfort and functionality. In several embodiments, a fluid sealing balloon/system replaces the pneumatic sealing balloon 260. Moreover, because the volume required to seal the pneumatic sealing balloon 260 is small, a compressor (or fluid pump) could consist of a flexible diaphragm driven by a servo (or servomechanism) or solenoid.

In several embodiments, the pneumatic sealing balloon 260 is transferred to the other nostril. However, in several embodiments, the system is bifurcated such that there are two separate nasal cups, with used fluid draining through them into separate egress tubes that join to form a common egress tube leading to the lower media reservoir 210.

As discussed herein, in several embodiments, heated air or other gas (in addition to or in place of fluid) can be delivered into the nose. Use of heated air presents certain advantages, such as, for example, air is supplied by the atmosphere, thus eliminating the need to supply it and, in several embodiments, can be ejected into the atmosphere, thus eliminating the need for a specific drain or spent fluid reservoir. Use of liquid also presents several advantages. For example, the heat capacity of a liquid, such as water, is greater than that of air (compare air's specific heat capacity by mass of 1.012 and a volumetric heat capacity of 0.00121 as compared to water's mass specific heat capacity of 4.1813 and volumetric heat capacity of 4.1796). Thus, in several embodiments, the use of a liquid improves (e.g., reduces) the time to transfer heat to the nostril (and thus the bacteria within) and thus achieves the decolonization treatment in a shorter time frame. In several embodiments, this also eliminates the need to use air heated to a greater temperature (e.g., above 122 F, such as for example, about 122° F. to about 125° F., about 125° F. to about 130° F., about 130° F. to about 140° F., about 140° F. to about 145° F., about 145° F. to about 150° F., and overlapping ranges thereof) and thus reduces the risk of burns with increased air temperature and inaccuracies in the detection of the target temperature at the nasal lining.

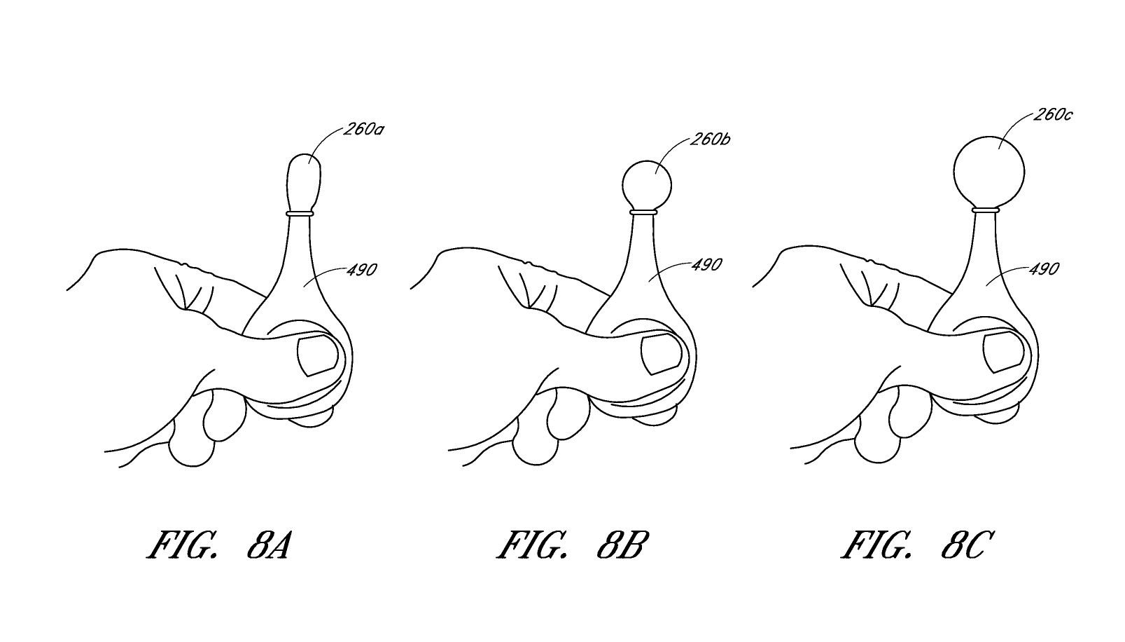

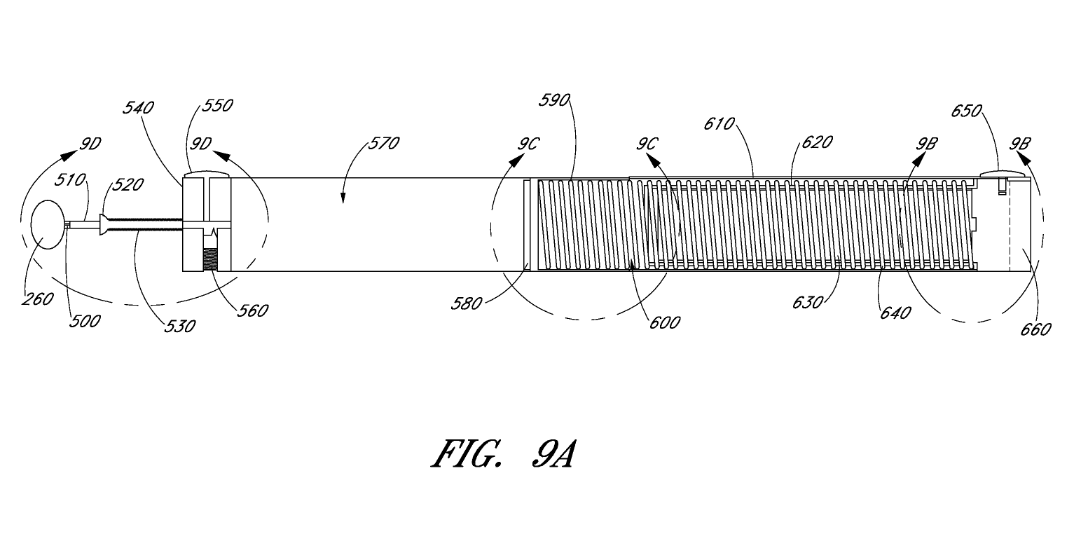

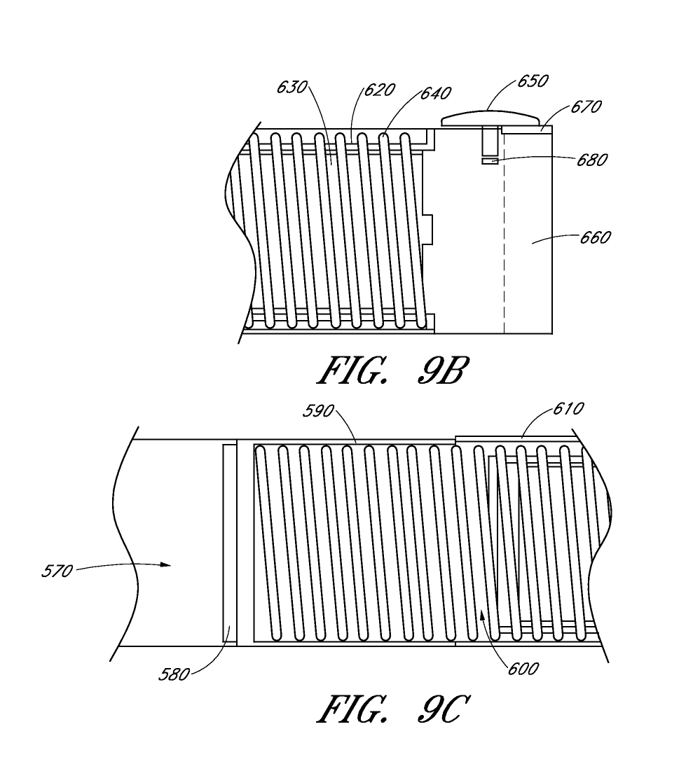

Alternatively, in several embodiments a self-contained, portable thermal decolonization device is employed to use heated liquid to decolonize the nasal cavity. One such device is schematically shown in FIG. 9A. The device comprises a proximal and a distal end. Beginning at the proximal end, the proximal end comprises electronic components and circuitry 660 that are activated by and “On/Off” button 650. A pressurization spring 640 lies in the proximal portion of the interior of the device, but outside the battery 630, which is housed within a battery holder 620. A piston retention cable 610 is cut by a knife contained within the “On/Off” button 650. In several embodiments, the “On/Off” button has multiple detents, for example a first detent to initiate heating of the decolonization media chamber 570 by the heater 580 and a second detent to cut the piston retention cable 610. In other embodiments, the “On/Off” button has a single “On” position that accomplishes both of the above procedures. A used fluid space 600 lies in the mid-portion of the device and functions to receive fluid that has exited the nasal cavity of a subject. A piston 590 is positioned distal to the pressurization spring 640. Upon actuation of the “On/Off” button 650, the piston retention cable 610 is cut and allows the pressurization spring 640 to provide force on the piston that (upon opening of a distal valve) causes the piston 590 to be translocated in a proximal to distal direction. More distal to the piston is a heater 580 that heats (upon activation of the device), operable to heat decolonization media. The distal region of the device comprises a dispensing button that allows the expulsion of the decolonization media out of the device. A valve spring 560 maintains the ingress fluid conduit 510 closed until a threshold pressure is reached. The ingress fluid conduit 510 is surrounded by a nostril cup 520 that is joined to a flexible accordion structure 530. As above, the nostril cup 520 functions to seal the nostril such that the decolonization media is sufficiently distributed throughout the nostril of the subject. The distal most portion of the device comprises a sealing balloon 260 that seals the anterior portion of the nostril from the posterior portion such that decolonization media that flows out of the distal portion of the device via the flow pathway 500 treats only the anterior portion of the nostril.

Greater detail of the proximal end of the device is shown in FIG. 9B. The “On/Off” button 650, upon depression interacts with an “On/Off” switch 680, that initiates the heating of the decolonization media by the heater. In several embodiments, the battery holder also serves as the proximal guide of the pressurization spring 640.

In FIG. 9C, the piston 590 can have various configurations. In one embodiment, the piston can be made hollow. In alternative embodiments, the piston can be made solid. As shown in FIG. 9C, the piston is hollow in order to facilitate centering (and proper expansion) of the pressurization spring 640. In several embodiments, a hollow piston allows for reduced manufacture costs and also enables the use of a longer pressurization spring 640, which in several embodiments, allows a more constant spring pressure. In several embodiments, the heater comprises a resistive element (e.g., a resistive heater) that connects to the electronics 660. In several embodiments, the connection comprises a loosely coiled wire connection (not shown) to allow for the dislocation of the heater (due to piston movement) in a distal direction without loss of connectivity with the electronics.

In addition, several embodiments employ a temperature sensing device operating in conjunction with the heater and the electronics to regulate the heat applied to the decolonization media. In several embodiments, the temperature sensor (such as a thermistor, not shown) is located distal to the heater to improve temperature sensing accuracy. In several embodiments, the piston is guided by a secondary sleeve within the device of a slightly smaller diameter than the outer diameter of the device. In several embodiments, the inner sleeve is notched or channeled in order to generate an open space to accept lead wires to the temperature sensor (such as a thermistor) from the electronics. In several embodiments, the sleeve also serves as part of the egress conduit for channeling used decolonization media from the nostril cup back into the used fluid space path. In addition to, or in place of, channeling the inner sleeve, in several embodiments, space for wires or fluid channels could be provided by evaginating at least a portion of tube's cross-section (e.g., providing an external conduit). Additionally, space for wires or fluid channels is optionally provided by using a piston having an elliptical cross-sectional shape with space for wires or fluid channels provided in the thicker portion of the elliptical sleeve surrounding the piston. In still additional embodiments, a round piston is used, and the outer casing is laterally extended, forming an ellipsoid shape, with space for wires or fluid channels provided in the thicker portions of the ellipse.

As shown in FIG. 9D, the distal end of the device comprises a series of accordion pleats 530 that enable the proper location of the nostril cup 520 to seal the outer portion of the patient's nostrils. To provide an egress fluid conduit, the internal diameter of the accordion pleats 530 is larger than the external diameter of the ingress fluid conduit 510. Thus, the space between these two features serves as a fluid conduit to allow fluid flow from the nostril of the user, between the accordion pleats 530 and the ingress fluid conduit 510 and to the used fluid space 600. The accordion pleats 530 need not have an accordion shape, but rather, depending on the embodiment, can comprise any flexible material that allows movement and positioning of the nostril cup 520. For example, rubber, nylon, or various polymers are used, depending on the embodiment. In several embodiments, series of longitudinal ribs or segments with flexible portions between them are provided to maintain the patency of the conduit for used decolonization fluid (while still providing the ability to place the nostril cup. As the piston is displaced in a proximal to distal direction, the used fluid space 600 increases in volume, allowing for sufficient volume to receive the used fluid. While not shown, in several embodiments the fluid conduit connecting the accordion pleats 530 and the used fluid space 600 extends through the valve assembly 565 to another notch on the external surface of the secondary sleeve mentioned above. In additional embodiments, other passageways for used decolonization media are generated (e.g., through other portions of the housing, etc.).

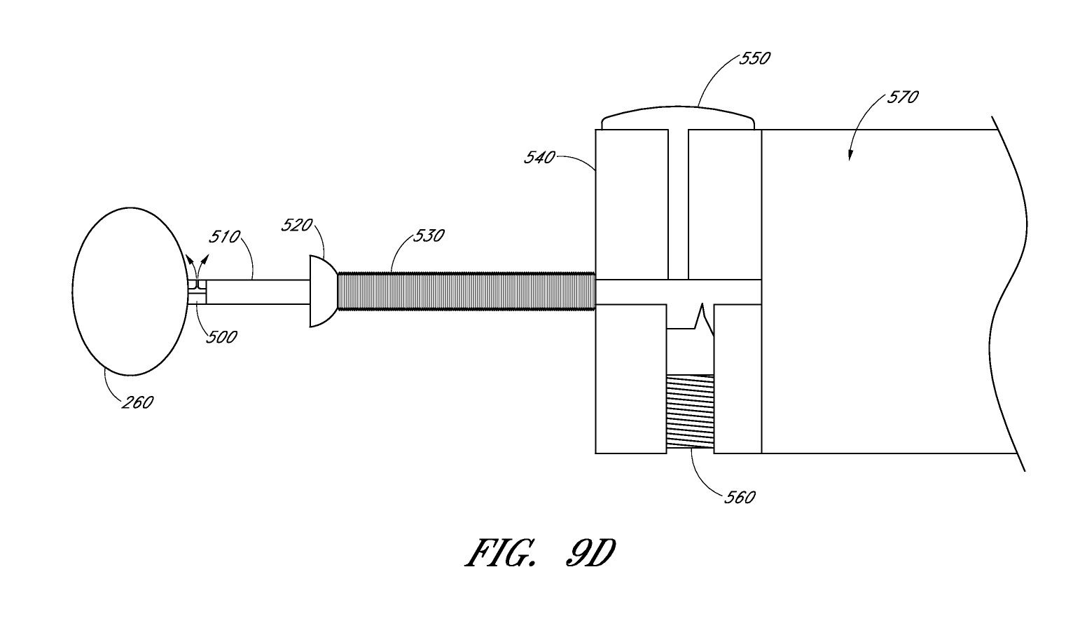

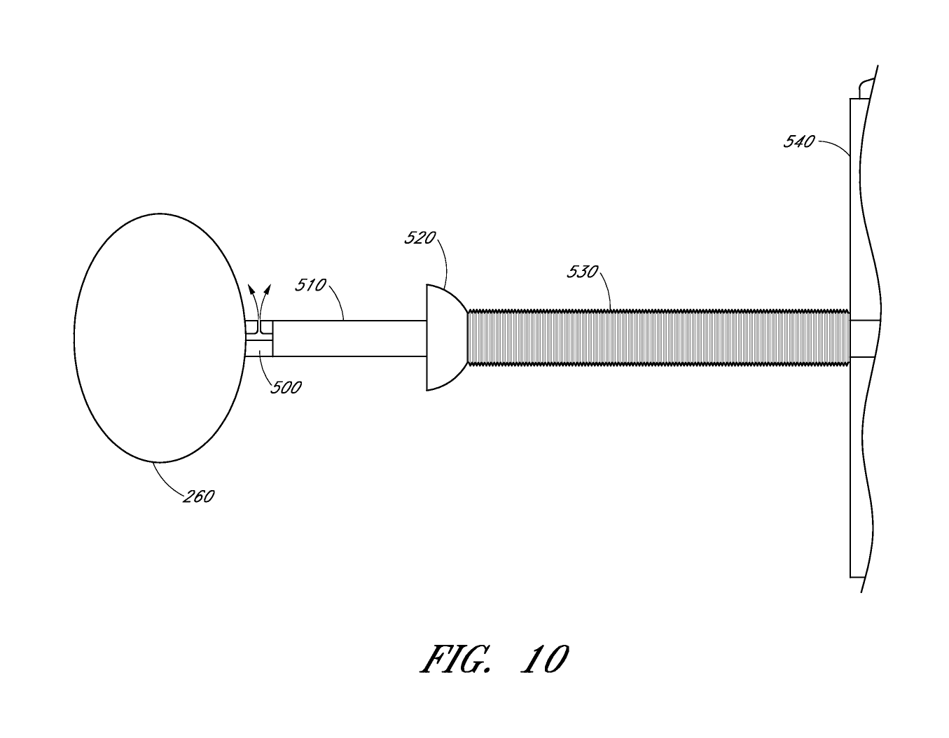

FIG. 10 provides a view of the distal-most region of the device for additional detail. The fluid flow out (arrows) of the ingress conduit 510 would result in fluid being deposited into the anterior nasal cavity (the posterior portion being sealed off by the sealing balloon 260). Fluid flow out of the ingress conduit 510 is regulated, in several embodiments by a valve 500. In several embodiments, the valve opens in response to a certain pressure, while in other embodiments, the opening of the valve is controlled by the electronics. In several embodiments the valve comprises a latching duckbill valve.

Some valves open in response to a positive pressure within the valve greater than their opening pressure and then close when the pressure within them falls below the opening pressure. Such valves are used in several embodiments. However, in several embodiments, the valves remain closed until a threshold opening pressure is reached, at which time they open and remain open (e.g., they latch open) even if the pressure within them falls below the opening pressure. FIGS. 11A-11C show one embodiment of such a valve.

FIG. 11A shows a duckbill valve in its closed position. The fixed portion of the valve 700 interacts reversibly with the mobile portion of the valve 690. In order to facilitate the motion of the valve, the valve comprises a flexible elastomeric polymer, in several embodiments. Suitable materials, include (depending on the embodiment) natural polyisoprene, synthetic polyisoprene, polybutadiene, chloroprene rubber, polychloroprene, Neoprene, baypren, butyl rubber, halogenated butyl rubbers, styrene-butadiene rubber, nitrile rubber, ethylene propylene rubber, ethylene propylene diene rubber, epichlorohydrin rubber, polyacrylic rubber, silicone, fluoroelastomers, perfluoroelastomers polyether block amides, polyethylene, ethylene-vinyl acetate and the like. In several embodiments the two portions of the valve are made of the same material, while, in some embodiments, different materials are employed. In several embodiments, the moveable portion 690 is molded and positions such that it is stretched beyond its resting position before the opening pressure is reached. At the time which the opening pressure is reached, that tip recoils back (secondary to elastic force) to the more proximal position shown in FIG. 11B (shown by arrow in 11A). Thus, as a result of the opening pressure, the moveable portion becomes stretched upward (as shown in FIG. 11B) in response to the opening pressure, but it cannot return to the closed position of 11A because the valve has recoiled to its resting position (FIG. 11B). FIG. 11B shows the valve in its open position. FIG. 11C shows the valve rotated 90° so the view is down the valve bore. The hatched tip of the valve in FIG. 11A corresponds to the hemispherical open area at the bottom of the valve in FIG. 11C. Optionally, the tip of the valve is positioned to rest in a small recessed groove (FIG. 11B) in the lower section of the valve 700. In such embodiments, this configuration provides extra resistance to extreme pressures that might otherwise cause the valve to shut.

The valve shown in FIG. 11 illustrates a valve with a single tooth, however in other embodiments, multiple tooth valves are used. In some such embodiments, a first (or multiple) tooth is compressed proximally and a second (or multiple) tooth is stretched distally before the opening pressure is reached, at which time each tooth recoils back to its resting position. In several embodiments, the bore of the valve is hemispherical, circular, or other convenient shape that provides sufficient fluid flow through the valve and also provides sufficient mechanical malleability to move in response to pressure.

Presented herein is one embodiment of the operational sequence performed to utilize a device as disclosed above for the thermal decolonization of the nasal cavity. In several embodiments, the use of the device disclosed above commences with the user pressing the “On/Off” button 650. This activates the electrical “On Switch” 680 and causes the knife on the button 650 to slice through the proximal piston retention cable 610. This embodiment utilizes on-demand mechanical pressurization, as the pressurization spring 650 then pressurizes the decolonization media 570 that cannot yet exit the device because the distal valve 560/565 is not yet open. Alternative embodiments utilize an electronic motor (or other alternative) to pressurize the decolonization media by advancement of a secondary piston or the like, electronically activated in response to the activation of the “On Switch” 680.

After activation, the electronics 660 supply power to the heater 580 to heat the decolonization media 570 to the treatment temperature as determined by feedback from a temperature sensor, such as a thermistor (not shown). Once the treatment temperature is reached, the user is prompted (e.g., by a light, for example an LED, beep, vibration, or some combination thereof) to insert the distal sealing balloon 260 into the nose slightly beyond the nasal vestibule. After insertion, the user presses the dispending button 560. This compresses the valve spring 560 and opens the valve in the valve assembly 565.

The pressurized decolonization media 570 travels through ingress conduit 510 to the sealing balloon 260, which fills, thereby expanding it to separate the anterior and posterior sections of the nasal cavity. Optionally, depending on the embodiment, a different mechanism is used to inflate the sealing balloon 260 (e.g., pneumatic pressure). In several embodiments, the sealing balloon is configured to limit the exertion of pressure on the interior portions of the nasal cavity (e.g., to limit pressure-induced discomfort). In several embodiments, the balloon has a certain thickness that, while allowing for expansion and sealing of the anterior cavity, does not produce undue pressure against the inner surface of the nasal cavity. Once balloon expansion is complete, continued distal movement of the piston results in continued movement of decolonization media, until such time as the valve assembly increases fluid pressure until it reaches the opening pressure of the valve assembly 500. As discussed above, the threshold pressure, once reached, induces the motion of the mobile portion 690 of the valve relative to the fixed portion 700, and the associated opening (and locking open) of the valve and flow of decolonization media out of the device and into the nasal cavity. In several embodiments, once the supply of decolonization media is exhausted (e.g., all media has been expelled from the decolonization media chamber 570) pressurization of the sealing balloon 260 ceases and the decolonization media within the balloon drains back into the nasal cavity (and then into the used media chamber). In several embodiments, once the supply of decolonization media is exhausted, the user is alerted, and thus is prompted to deflate the sealing balloon. Thus, deflation of the sealing balloon, depending on the embodiment, can either be automated or manual.

The self-contained nature of several embodiments of the devices is advantageous in some cases. For example, in several embodiments this improves the ability of the device to be sterilized and re-used (although in some embodiments the devices are disposable). In some embodiments, the devices are not self-contained, but rather are modular. For example, in several embodiments, the device does not include (at least in the structure of the device itself) one or more of a heater, battery, battery holder, thermistor, electronics, internal wires, on switch (and its knife), pressurization spring, piston retention cable, and secondary sleeve. In such embodiments, those functions or features are performed by accessories in a system (e.g., a base station or accessory pack).

As use of gas (such as air) and liquid (such as a saline, i.e. a saline solution in water) both present certain advantages when decolonization is desired, in several embodiments, there are also provided devices for the use of heated air to decolonize the anterior portion of the nasal cavity.

For example, while conformable liquid tips are used in several embodiments, heated emission of air is used in several embodiments. In several embodiments, variable air pressure and/or vacuum mechanisms are used to tailor thermal plume propagation, and in several embodiments, enhance treatment comfort. Heated emission of air is also used, in several externally applied embodiments, such as, for example, a heavily crusted and/or irregular surface.

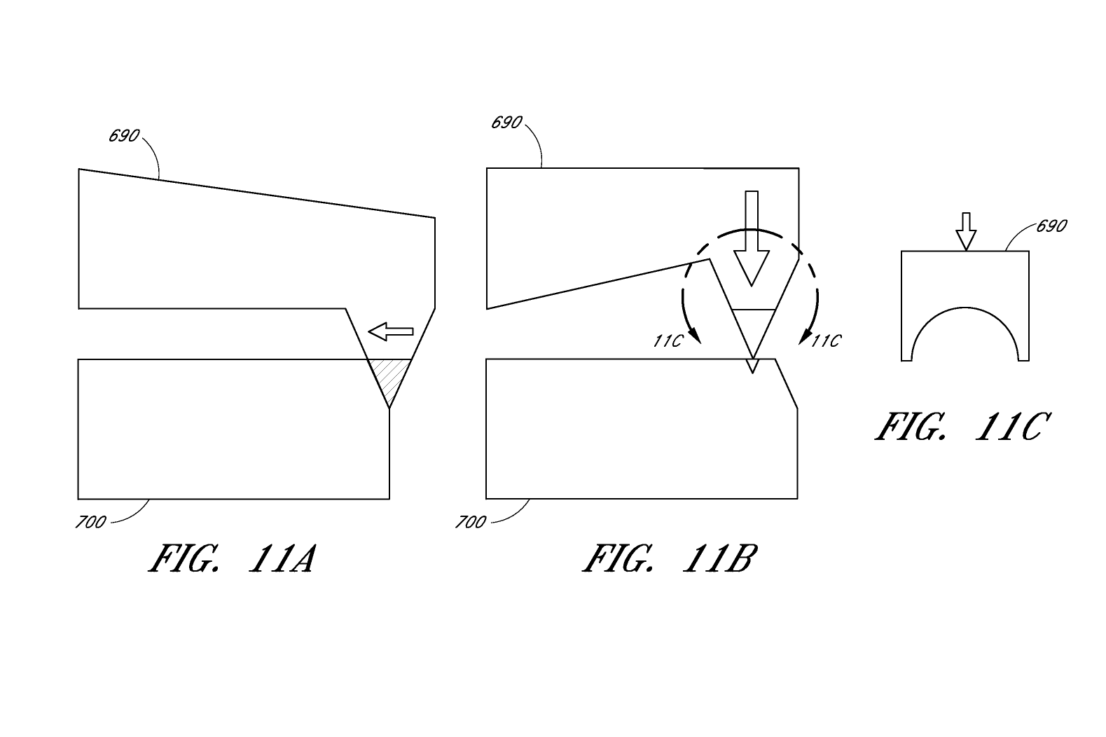

Thus, in several embodiments, there is provided a device for the decolonization of a surface using heated air. As represented in the embodiment shown in FIG. 12, the device 710 comprises an elongate member with a proximal and distal end. The proximal end of the device houses a battery and associated device electronics 850. In some embodiments, however, the device is externally powered (e.g., via a plug-in cord, etc.).

In several embodiments, the electronics further comprise a barcode scanner to read a patient ID bar code and/or an employee's ID bar code and transmit/record data documenting the treatment.

Air is drawn into the device via a fan 800 (driven by a fan motor 810). In several embodiments, the air is filtered as it enters the device (or at another stage prior to being propelled to the surface to be decolonized). In several embodiments, the filter is to remove gross impurities in the air (e.g., dust, pollen, etc.). In several embodiments, the filter is multi-stage, with one stage optionally comprising a HEPA filter. The fan passes the air through a heating unit 770 and through a switching valve assembly 760 and out of the distal end of the device via the probe tip 720. An optional vacuum motor 840 brings air back from the nasal cavity via the vacuum return 830 and expel the air out a side port back into the atmosphere (not shown). Optionally, the side port also comprises a filter. The electronics control the fan motor speed and vacuum motor speed to achieve the desired thermal plume propagation.

As many locations providing decolonization treatment have pre-existing compressed air and vacuum sources, the fan and fan motor, and/or vacuum pump, could be eliminated, and replaced by valves regulating air flow and vacuum. In the version that emits via side ports only (described in more detail below), the vacuum pump (or source) is optionally eliminated.

In several embodiments, the device further comprises a temperature sensor (such as a thermistor) 750 which senses air temperature and provides feedback to the electronics to regulate air temperature to a desired set point. The switching valve assembly 760 functions, in several embodiments to alternately expel heated air from the probe to heat the nasal epithelium and to receive the air and bring it back into the device. In several embodiments, the probe comprises a plurality of ports (e.g., port in the distal-most portion of the probe and one or more side ports). In several embodiments, the probe is sealed from the device with O-rings or another appropriate seal (730a and 740). Depending on the embodiment, the switching valve may also be used with liquid media, air (as discussed above) or any fluid that us used for decolonization according to the embodiments disclosed herein. In several embodiments, the frequency of operation of the switching valve is regulated by the treatment time required at one target treatment area. Treatment time, in several embodiments, depends on the decolonization media used (e.g., air or liquid), its temperature, its humidity, and/or its flow rate. In some embodiments, several seconds (e.g., about 3 to about 5 seconds, about 5 to about 10 seconds, about 10 to about 15 seconds, etc.) are necessary to raise, for example, the nasal lining to the target temperature. In several embodiments, longer treatment times are used, for example, about 3 seconds to about 120 seconds, including about 3 to about 30 seconds, about 30 to about 60 seconds, about 60 to about 90 seconds, about 90 to about 120 seconds, and any time in between those ranges. Additionally, in some embodiments, treatment times on the order of minutes are employed, such as, for example, about 1 to about 2 minutes, about 2 to about 3 minutes, about 3 to about 4 minutes, about 4 to about 5 minutes, or longer (and including any time in between those ranges. In one embodiment, the switching valve assembly alternately expels heated air from the probe's apical port and side ports to enable simultaneous heating of nasal epithelium adjacent to the probe's apex and sides. The valve simultaneously switches between the heated air and vacuum so ports can expel air or ingest it, respectively. In several embodiments, the at approximately 50% through the total treatment time (which again, varies depending on the target tissue, the degree of colonization, etc.), the valve switches to heat different nasal areas, targeting the area beyond the apical port or those adjacent to the side ports. Thus, in several embodiments, the valves need switch only once per treatment session. However, in some embodiments, the valves switch 2, 3, 4, 5 or more times, in order to treat multiple smaller regions within a target tissue area. In some embodiments, the switching results in intermittent heating of an area (e.g., a first area is heated, the switching valve switches and that area is no longer heated, and then the switching valve switches again, heating the first area a second (or more) time).

Humidified air has a greater heat capacity, and thus, in several embodiments, the device comprises a hygrometer 780 that detects the amount of moisture in the air to be emitted. Through the use of a humidifier 790 and a water tank 820, the humidity of the emitted air can be adjusted, depending on the conditions of colonization of a particular subject and the ambient humidity. In such embodiments, the hygrometer provides feedback to the electronics to control the humidifier so the emitted air is at the desired humidity set point (which, in several embodiments, ranges from about 80 to about 90%, about 90 to about 95%, about 95 to about 99%, or closer to 100% relative humidity). The relative humidity would drop as the air passed through the heater; with a fixed amount of water vapor in a given air mass, increasing its temperature increases its capacity to hold water, thereby lowering its relative humidity). In several embodiments, one or more ultraviolet LEDs (or other light source) or other sterilization approaches may be used to maintain water sterility in the water tank.

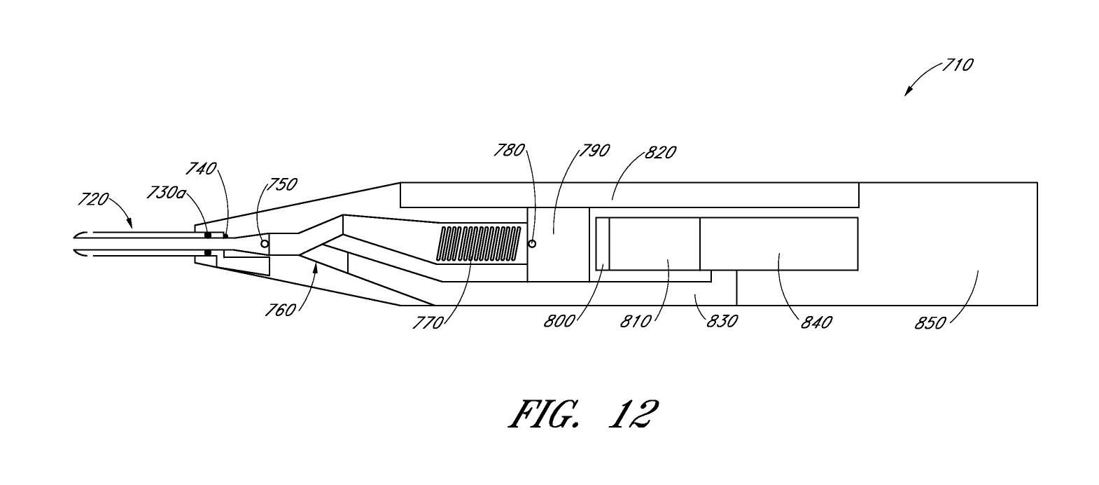

The switching valve assembly switches the heated and vacuum air, as shown schematically in FIGS. 13A-13B. As shown in these Figures, the point labeled H is permanently connected to the heated air source and the point labeled V is permanently connected to the vacuum return line. The transition of the switch 860a from its position in FIG. 13A to 860b in FIG. 13B alternates port function so the distal-most port emits or ingests air as the side ports ingest or emit air, respectively. In several embodiments, the valve assembly is configured to allow laminar or substantially laminar air flow.

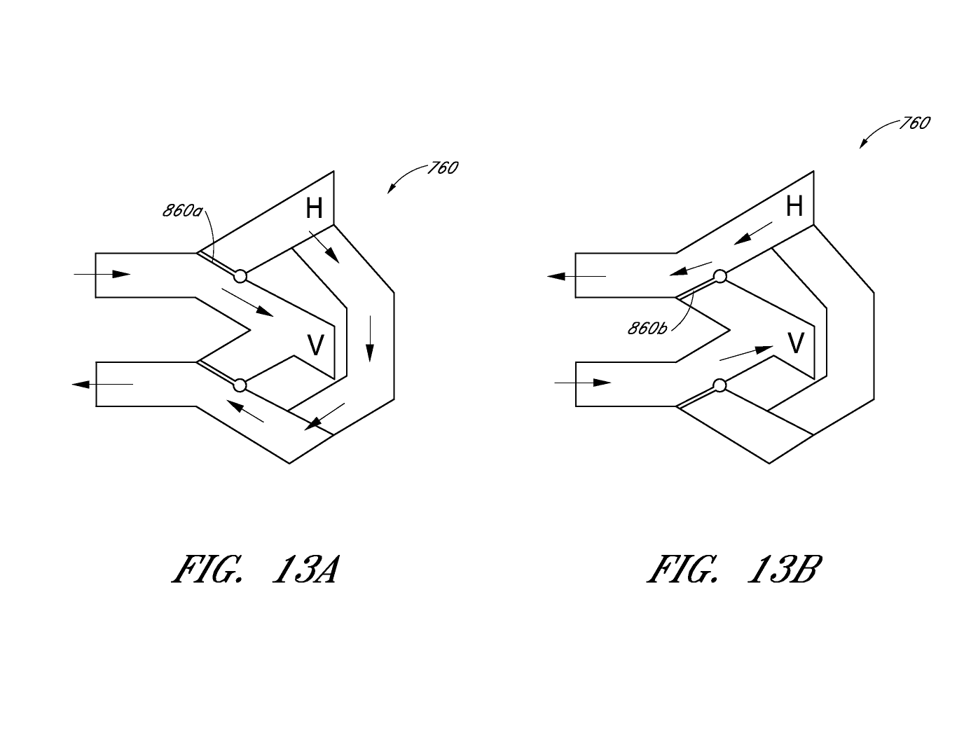

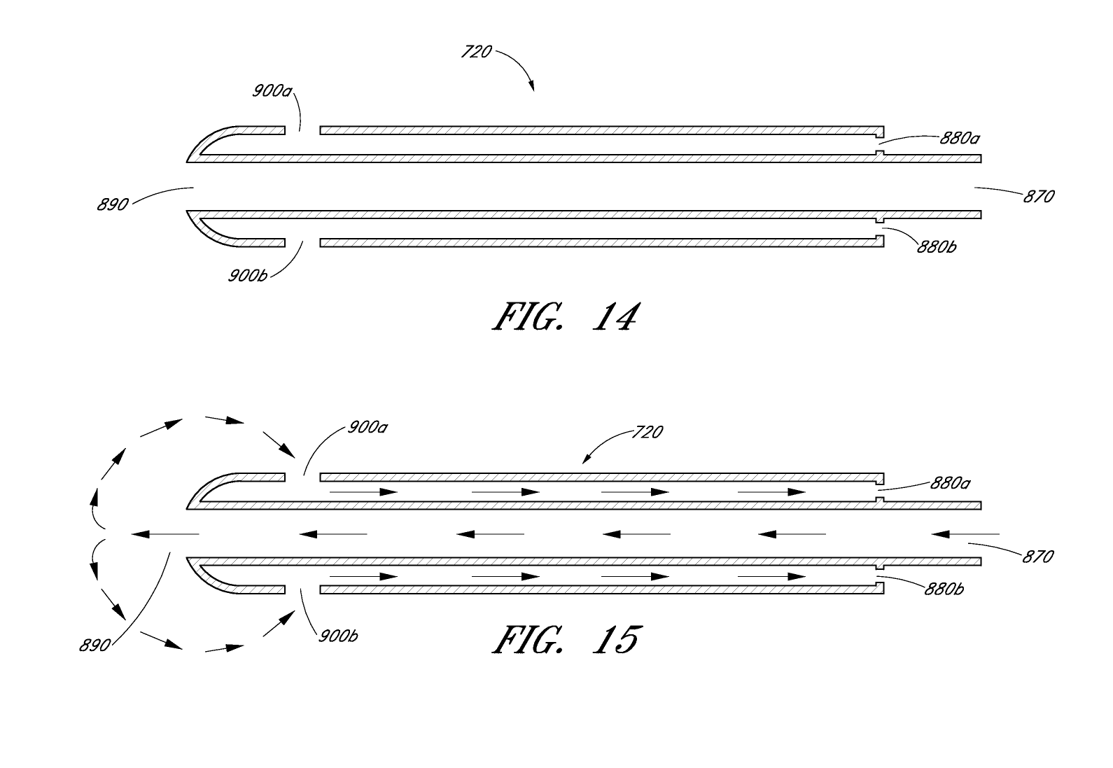

FIG. 14 depicts a more detailed schematic of one embodiment of an air emitting probe 720. In several embodiments the air emitting probe comprises a proximal aperture 870 configured to interact with the valve assembly described above and receive air that has been heated to a temperature sufficient to decolonize the nasal epithelium. The proximal aperture is in communication with the distal port 890, which, in several embodiments expels the heated air into the nasal cavity. Lateral ports 900a and 900b, are positioned near the distal end of the probe. Depending on the embodiment, the position of the lateral ports may be varied based on, for example, the dimensions of the nasal cavity of the subject to be treated, the degree of colonization/decolonization (e.g., severity of infection), the anticipated temperature and/or air flow to be used, among other considerations. Thus, in several embodiments the lateral ports are more distal than pictured, while in other embodiments, they are more proximal. Moreover, in certain embodiments, a plurality of lateral ports exist in the probe. The lateral ports are in communication with lateral flow pathways 880a and 880b, which are in further communication with the vacuum return. FIG. 15 depicts one embodiment of air flow through the probe. In alternative embodiments, the air can be directed out of the lateral ports 900a and 900b, and return into the probe via the distal port 890 (e.g., flow reversed from that depicted in FIG. 15).

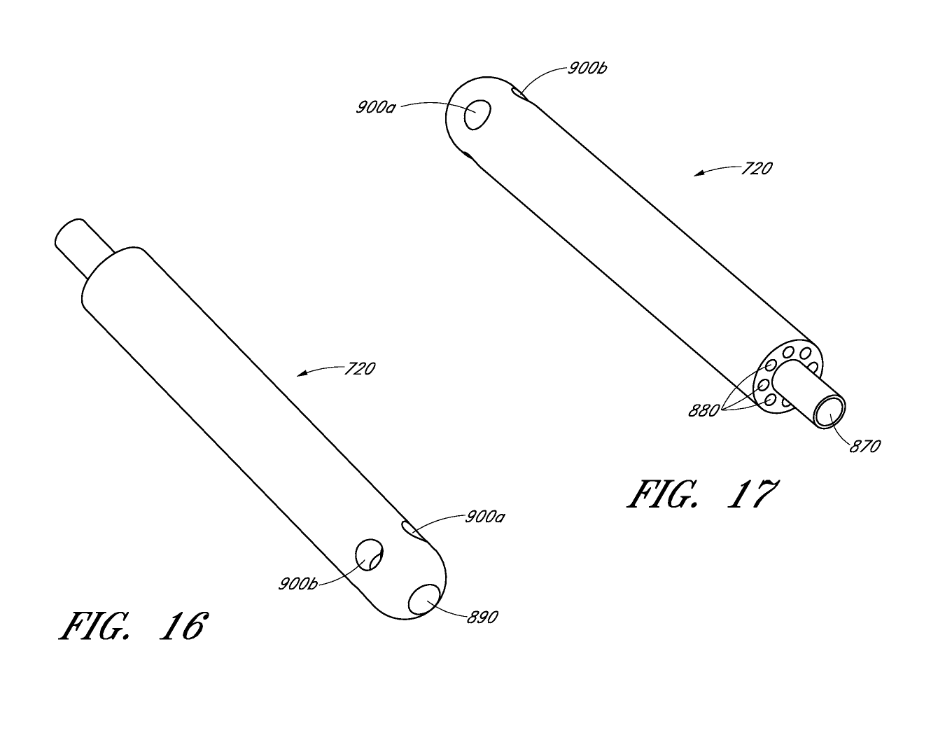

FIGS. 16 and 17 depict additional features of certain embodiments of air emitting probes. FIG. 16 depicts an oblique view of a probe comprising a distal port 890 and two lateral ports 900a and 900b. FIG. 17 depicts a rear oblique view depicting a plurality of lateral flow pathways 880. As discussed above, depending on the embodiment, the number of lateral flow pathways and lateral ports may vary depending on the embodiment. Some embodiments have only a single lateral port and lateral pathway, whereas other embodiments, have at least two lateral ports and lateral pathways. In some embodiments, the probe comprises between 4 and 8, between 5 and 9, between 6 and 10, between 7 and 11, between 8 and 12, between 9 and 13, between 10 and 14, between 11 and 15, between 12 and 16 (and overlapping ranges thereof) lateral ports and flow pathways. In several embodiments, the side ports are equipped with port guards that space the probe away from the nasal epithelium (e.g., to avoid vacuum suction directly on the tissue). However, in some embodiments, the guards are optional, such as when the side ports are only to be used for emission of heated air.

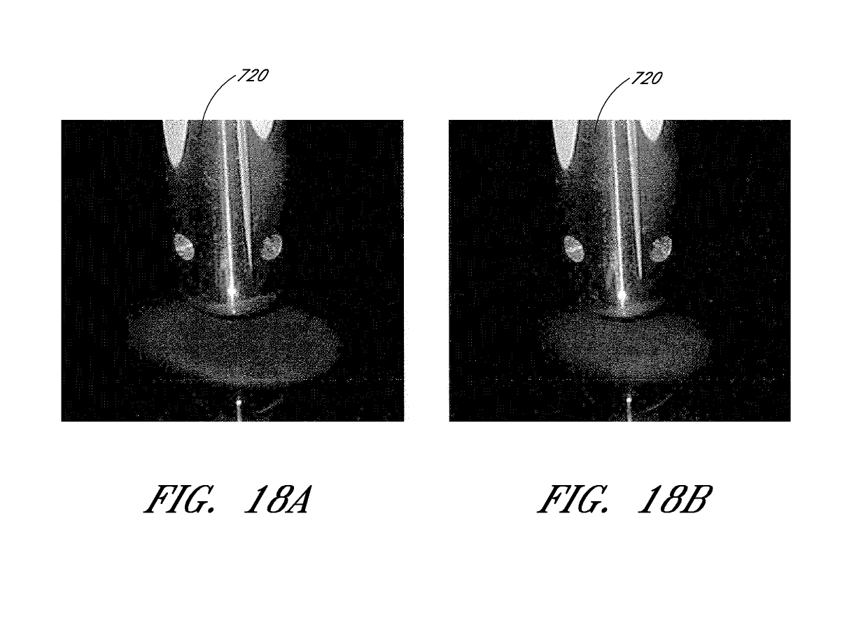

The configuration of the ports, in several embodiments, is configured in a manner such that the plume propagation of the emitted air is controlled to a desired pattern. FIG. 18A depicts a more diffuse, intense heating effect while FIG. 18B depicts a more concentrated, less intense plume (as shown on the thermochromic film below the probe).

The material of the probe is varied, depending on the embodiment. For example, in several embodiments the probe is machined from metal, such as titanium, gold, silver, platinum, other metals and alloys. An example probe may comprise metal and one or more other materials. An example probe can be formed from, or otherwise comprise, a polymer, such as polypropylene, polyimide, polyvinyl alcohol, polyvinyl pyrolidone, biopolymer (such as collagen, or chemically-treated collagen), polyethersulfone (PES), poly(styrene-isobutyl-styrene), polyurethane, ethyl vinyl acetate (EVA), polyetherether ketone (PEEK), fluoropolymer (such as Kynar (Polyvinylidene Fluoride; PVDF), or Polytetrafluoroethylene (PTFE)), Polymethylmethacrylate (PMMA), Pebax, acrylic, polyolefin, polydimethylsiloxane and other silicone elastomers, polypropylene, elastomers, thermopolymers, other plastics, and mixtures or combinations thereof. Example probes may comprise minerals (such as apatite, hydroxyapatite, and the like), ceramics, glass, shape memory materials (such as nitinol), and mixtures or combinations thereof. Additional suitable materials used to construct certain embodiments of the probe include thermoplastic polyurethanes, silicone-modified polyether urethanes, poly(carbonate urethane), or polyimide. Thermoplastic polyurethanes are polymers or copolymers which may comprise aliphatic polyurethanes, aromatic polyurethanes, polyurethane hydrogel-forming materials, hydrophilic polyurethanes, or combinations thereof. The probe may be flexible or rigid, or include a combination of flexible and rigid portions.

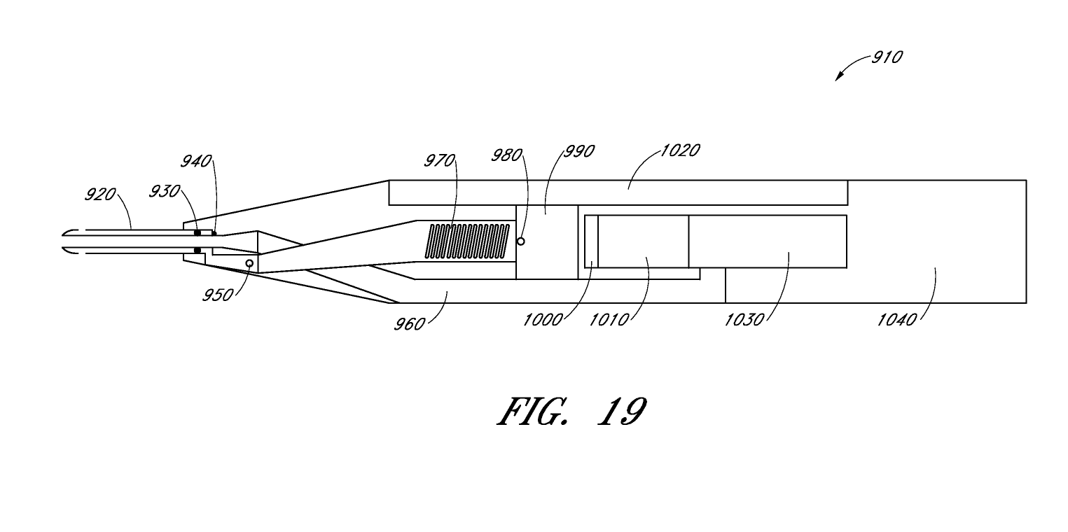

Alternative embodiments of the device are used, depending on the degree and location of colonization. For example, in several embodiments, colonization may be significantly more posterior in a first individual as compared to another. Nasal mapping is therefore used, in several embodiments, prior to decolonization. Staph nasal mapping is achieved, in one embodiment, by swabbing specific nasal areas in each patient sampled, carefully avoiding contact with the anterior nares (or its hair) by using a speculum for sampling transitional epithelium and deeper areas. If deep colonization is detected, the device described above (e.g., a distal port and side ports) would be used, as the distal port would emit air deeper into the nasal cavity. If deep colonization were not significant, alternative embodiments of the device could be used. For example, in such cases, the device could emit heated air laterally only. In such embodiments, port guards and the switching valve assembly that otherwise would alternate the apical and side port function (emitting air versus ingesting it) are optionally eliminated. FIG. 19 shows one embodiment of such a modified device 910 (which is otherwise as described above, but for the absence of the switching valve assembly).

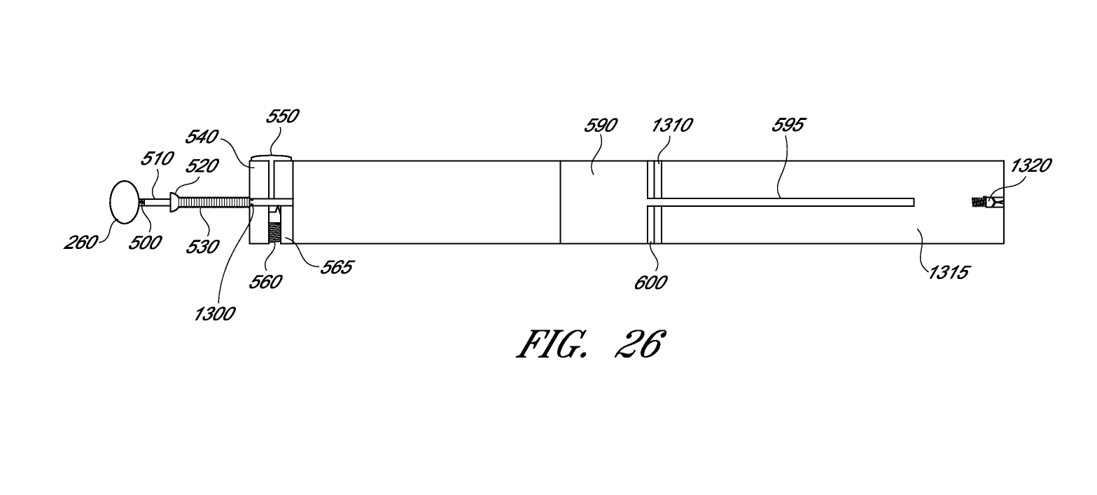

In additional embodiments, the decolonization media delivery device is not “self-contained”, but rather is configured to reversibly interact with a base station that performs certain functions or steps of decolonization media delivery. In several embodiments, such a device configuration may comprise a device that does not include one or more of a integral heater, battery, battery holder, thermistor, electronics, internal wires, on switch, pressurization spring, piston retention cable, or secondary sleeve. Rather, in such embodiments, a base station performs on or more of those functions. In several embodiments, such device embodiments reduce per patient cost and reduce the risk of cross-contamination. One embodiment of such a device is shown in FIG. 26. FIG. 26 depicts a device comprising a distal end as discussed above (e.g., a sealing balloon 260, a duckbill valve 500, a fluid conduit 510 surrounded by a nostril cup 520 that is joined to a flexible accordion structure 530. The proximal portion of the device comprises, in several embodiments, a charging valve assembly 1320 that allows compressed gas (e.g., air, nitrogen, carbon dioxide, or combinations thereof) through the charging valve assembly into the gas cylinder 1315. Once the gas cylinder is sufficiently charged, pressure can be applied to the free end of the piston rod 595. Depending on the embodiment, the diameter of the piston rod can be varied, in order to vary the force transferred to the piston, and concurrently the pressure applied to the decolonization media. Such embodiments are advantageous, for example, for tailoring a device for use with an adult versus use with a child, wherein the target orifice is of different dimensions and/or volumes. In several embodiments, the device comprises a fixed divider 1310, which separates the gas cylinder 1315 from the decolonization media 570. The fixed divider may optionally include an O-ring (or double O-ring or other sealant mechanism) to prevent pressurized gas from leaking into the used media chamber 600.

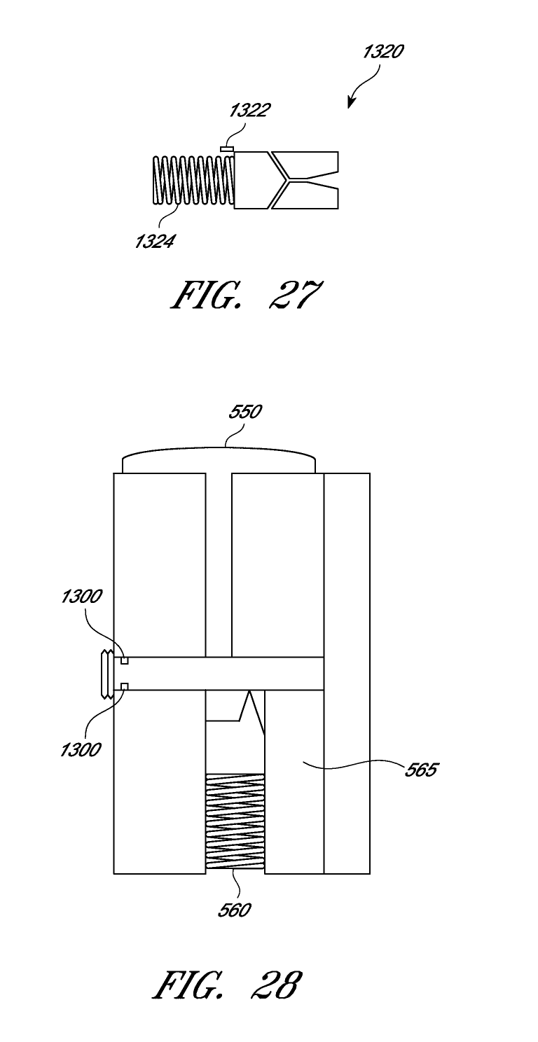

FIG. 27 depicts certain features of the charging valve assembly. The assembly is configured to reversibly interact with a charging nozzle from the base station via a port 1323 in the valve assembly. The pressure from the gas source results in temporary translocation of the piston 1325 from the valve seat resulting in compression of the valve spring 1324 and opening the piston port 1322. Opening of the port permits compressed gas from the base station to enter the gas cylinder. When pressurization is complete, spring pressure reseats the piston on the valve seat. Pressure from the gas cylinder acts on the base of the piston 1325 to encourage seating of the piston on the valve seat, thereby reducing leakage of compressed gas.

In several embodiments, such as that shown in FIG. 28, one, or a plurality of apertures 1300 are present along the ingress tube 510. In embodiments in which gas pressure is used, the pressure drops during delivery of the decolonization media, which decreases media flow rate. Depending on the embodiments, the aperture is adjustable to maintain media flow rate. For example, in several embodiments, the aperture comprises a solid (e.g., a salt) that is soluble in the decolonization media. In such embodiments, while the gas pressure is initially high, the salt aperture provides a constriction (reduced diameter) that reduces flow rate through the aperture. Gas pressure drops as the decolonization media passes into the patient (and returns to the used media space) but the flow rate remains stable as the salt within the aperture dissolves, enlarging its bore and thus reducing the constriction. In additional embodiments, mechanically adjustable, electrically controlled, or electroactive materials are used to adjust the flow rate in proportion to the gas pressure (or to spring pressure, in those embodiments employing spring pressure).

In additional embodiments, the delivery of the decolonization media need not employ compressed gas, but may employ a motorized unit to rotate the piston rod (threaded to engage the fixed divider) within a central bore of the fixed divider. The rotary motion moves the piston forward and dispenses the decolonization media. In such embodiments, the charging valve assembly is not required. In several embodiments the piston rod 595 is dimensioned such that it projects from the proximal end of the device, such that a user can identity when the piston is fully advanced. In several embodiments, this allows identification of the treatment endpoint. In still additional embodiments, the piston rod is dimensioned to minimally extend from the end of the handpiece and engage a complementary component that transfers linear motion to the piston via a connecting rod.

In several embodiments, placement of the device in the base station prior to use allows the base station to heat the decolonization media, for example by microwave radiation, electromagnetic induction, or conduction with heat transferred from a heated surface inside the base station. In several embodiments, the heating endpoint (e.g., the target temperature for the decolonization media within the device, which in some embodiments, is adjusted to account for possible heat loss during the delivery process) is sensed using a temperature sensor, such as a thermistor, in direct contact with the decolonization media. Alternatively, in several embodiments, the temperature can be detected by a noncontact infrared thermometer. In still additional embodiments, the heating endpoint is determined by storing the devices in a temperature-controlled compartment in or functionally associated with, the base station so the initial temperature of the decolonization media was known. Thus, in such embodiments, a microcontroller within the base station computes the energy required for the decolonization media to reach the endpoint temperature.

### Otic Thermal Decolonization

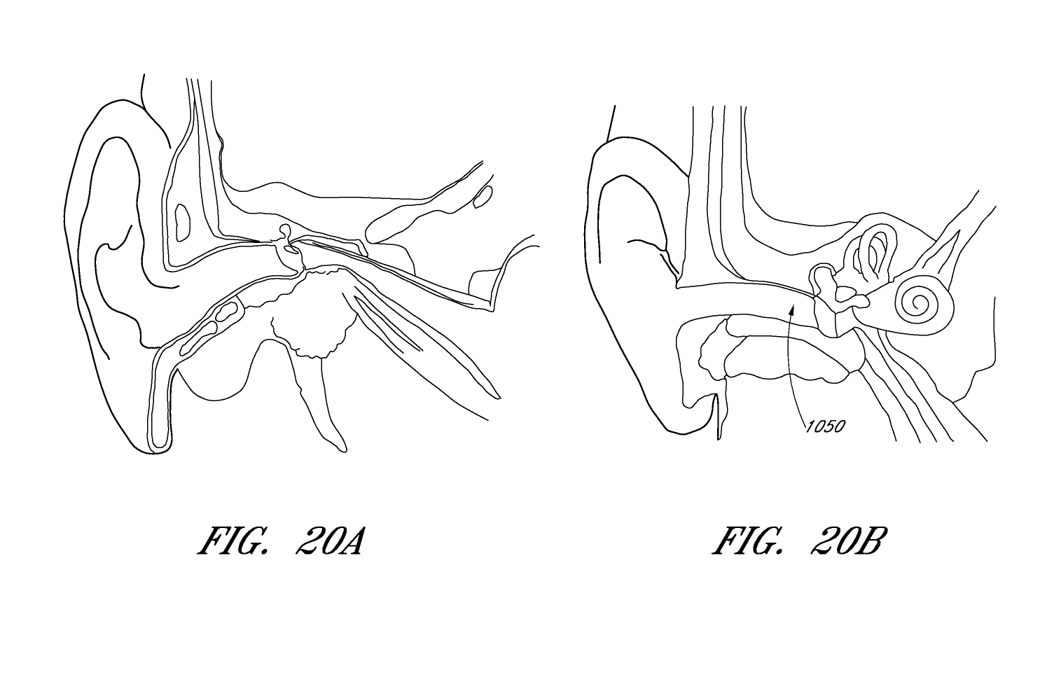

As discussed above, temperatures of approximately 120-125 degrees Fahrenheit can kill Staphylococcus aureus. Applying controlled heat can quickly inactivate and kill bacteria within the ear canal (schematically shown in FIGS. 20A and 20B, 1050), but heat reduces the density of endolymph within the semicircular canals (part of the vestibular system governing balance and spatial orientation), and once heat diffuses there vertigo, occasionally nausea and vomiting, and physiologic nystagmus can result. Thus, in several embodiments, the devices for otic thermal decolonization provide methods of controlling heat dissipation throughout the ear, and reduce adverse effects. For example, at intervals, a cool fluid may be used to remove heat from a body cavity such as the ear canal. In several embodiments, example devices and methods disclosed herein are also useful to remove or ameliorate buildup of earwax and/or pus secondary to ear canal infections (otitis externa). In such instances, example devices and methods may reduce risk associated with ear curettes or ear canal irrigation.

In several embodiments, thermal decolonization comprises infusion of heated fluid, such as a liquid (e.g., water, saline, or either or another vehicle; optionally including an added cerumenolytic agent) under controlled low pressure into the external auditory canal to inactivate Staphylococcus aureus within the ear canal. To reduce side effects (vertigo, nausea, vomiting, and nystagmus) secondary to semicircular canal endolymph density reduction, effluent liquid is collected. From its average temperature, its heat loss is easily calculated, thus indicating the amount of heat transferred into the ear canal. In several embodiments, the device calculates the approximate amount of cool fluid necessary to remove some or all of the heat energy gained by the ear canal. In such embodiments, cool fluid is instilled into the ear canal to isocalorically balance the heat gain and loss. In some examples, the heating and cooling aspects may be estimated using the initial temperatures of the fluids used, and these estimates used to obtain an approximate energy balance. Cool fluid has a temperature less than the decolonization fluid, and may have a temperature less than body temperature. Depending on the source of cool fluid, actual cooling may not be required. For example, cool fluid may be water that is heated to a temperature above room temperature but less than body temperature. The effluent cool fluid is optionally collected and combined with decolonization effluent fluid to permit a precise calculation of the net heat gain or loss into the ear canal. If needed, the device can then optionally instill additional hot or cool fluid into the ear canal to more precisely neutralize the applied heat and therefore avoid or greatly minimize side effects resulting from endolymph density changes.

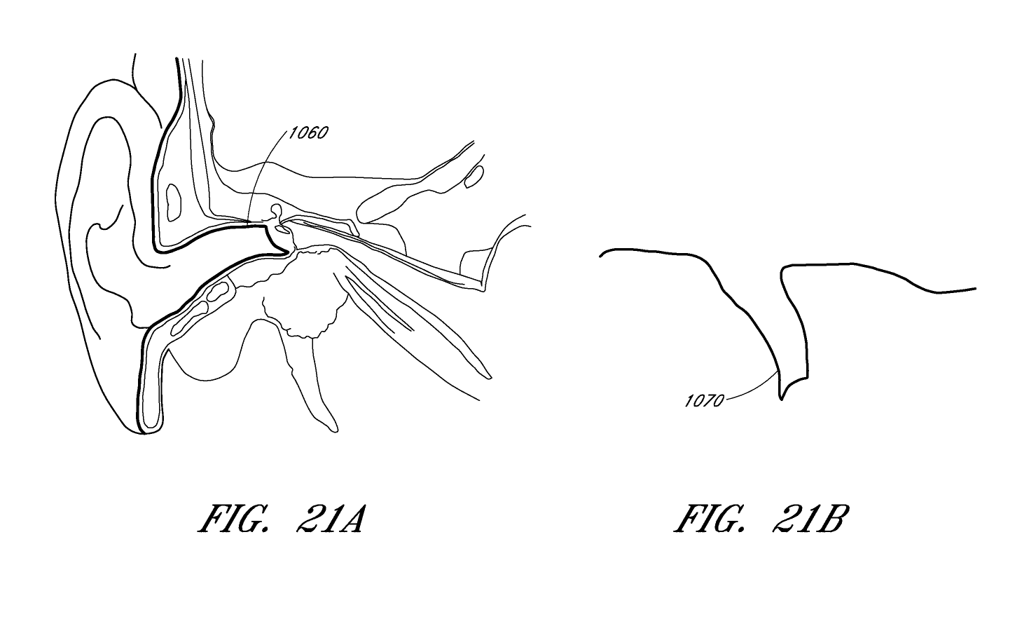

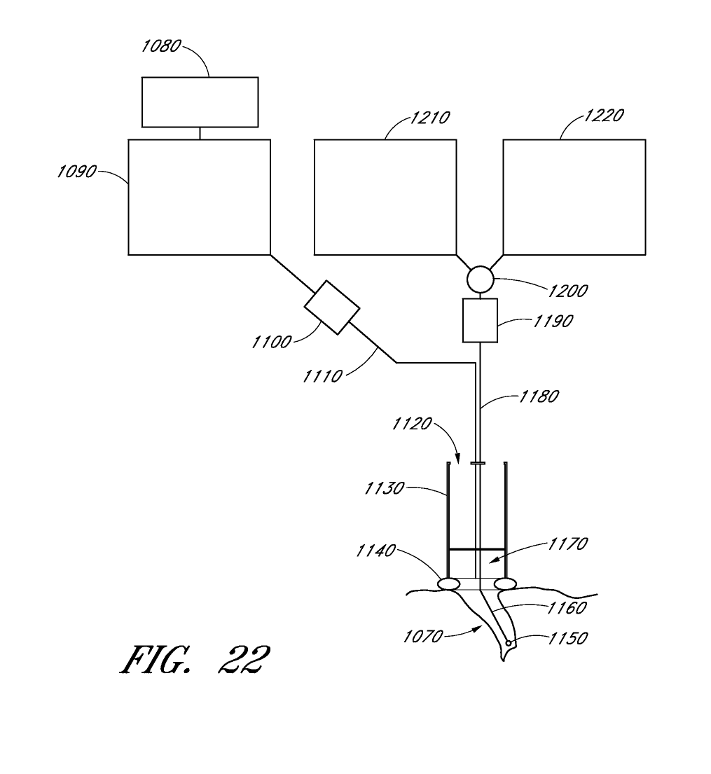

FIGS. 21A and 21B depict the ear canal schematically and the target region of treatment 1070; while FIG. 22 depicts one embodiment of a device for otic thermal decolonization. The device in FIG. 22 comprises, in several embodiments, a chamber for hot fluid 1210, a chamber for cold fluid 1220, a valve 1200 that allows for hot and cold fluid to be combined. In several embodiments, the hot chamber includes a heater, pump to circulate the fluid within that chamber (to accelerate heating, analogous to a convection oven) and to equally distribute the heat within the chamber. In several embodiments, the chamber further comprises a temperature sensor (such as a thermistor or other temperature sensing device) to sense the average fluid temperature. In several embodiments, the cold chamber includes a heater, pump to circulate the fluid within that chamber (to accelerate heating, analogous to a convection oven) and to equally distribute the heat within the chamber. In several embodiments, the chamber further comprises a temperature sensor (such as a thermistor or other temperature sensing device) to sense the average fluid temperature. The fluid may be a liquid, such as an aqueous liquid, such as water or an aqueous solution such as saline.

In some embodiments, treatment is provided when the subject is in a position other than with the ear canal facing upward. For example, in some embodiments, treatment is provided when the ear canal is facing downward (e.g., with the subject prone or positioned on their side). If the patient is sitting or standing, the head can remain in a neutral position or be tilted toward the treatment side. In such embodiments, fluid is able to exit the ear canal via gravity. To capture the used fluid so its heat loss can be measured for isocaloric calculations, as the application chamber 1130 extends outward from the auricle (pinna), it bends upward at an approximately 90° angle (e.g., for use with subjects who are sitting or standing), thus forming an “L” shape. Alternatively in several embodiments, for subjects on their side with the ear canal pointing downward, the application chamber 1130 extends outward from the auricle and bends at an approximately 180° angle (forming a “J” shape).

When the ear canal is blocked or substantially narrowed, in several embodiments the ingress probe 1160 is not used and fluid is pumped into the ear canal by the fluid ingress tube 1180 that ends just before entering the ear canal. In such embodiments, the distal pressure sensor 1150 is optionally eliminated. In several embodiments, one or more of the egress pump 1100 and the vacuum pump 1080 are eliminated, for example when the isocaloric chamber 1090 is positioned gravitationally below the ear so flow from it would result from gravity. In such cases, the egress tube 1110 enters the application chamber 1130 at the gravitationally lowest point of the application chamber, rather than parallel the fluid ingress tube 1180.

Depending on the temperature of the water fluid put into it, the cold chamber, in several embodiments, is configured to warm the water fluid with a heater or cool it with the cold face of a thermoelectric device (e.g., via the Peltier effect) until the water fluid reaches the target cold temperature. In several embodiments, the target temperature is approximately 70° F., though depending on the embodiment target temperatures can rang from between about 50° F. and about 60° F., between about 60° F. and about 70° F., between about 70° F. and about 80° F., and overlapping ranges thereof In several embodiments, the use of a thermoelectric device is advantageous because it can heat or cool depending on the polarity of the electric current fed to the device, thereby allowing it to function as cooler and heater. Thus, in several embodiments utilizing a thermoelectric device, a separate heater and cooler are not used.

In several embodiments, the device comprises a microcontroller to sense, integrate and adjust the temperatures of the fluids in the various tanks, vary the flow and pressure of fluid as needed, etc. In several embodiments, the microcontroller adjusts the valve to allow either the hot or cold fluid to be fed to the ingress pump 1190, which pumps fluid into the ear canal 1070 via a fluid ingress tube 1180. The fluid ingress tube proceeds into the ear canal, where the ingress probe 1160 is positioned close to the eardrum. The ingress probe, in several embodiments, may comprise a pressure sensor 1150 (discussed in more detail below). In several embodiments, the device comprises a conformable gasket (e.g., conformable silicone solid or pneumatic gasket) 1140 to seal the application chamber 1130 against the ear of the patient. The seal allows the ingress fluid (hot or cold, or a combination) to fill the fluid chamber 1170 (e.g., the generated by the sealing of the application chamber against the ear). In several embodiments, the cylindrical (in cross section) application chamber is held by the physician, nurse, or patient, or in some embodiments a strap or headpiece.

In several embodiments, the application chamber comprises a vent 1120, which allows fluid overflow and hence, avoids development of overpressure. An egress tube 1110 is configured to sit within the fluid chamber 1170 and withdraw the fluid that has been emitted by the ingress probe. The egress tube is in communication with an egress pump 1100 that pumps the fluid out of the ear canal and, in conjunction with a vacuum source 1080, into an “isocaloric” chamber 1090. As discussed above, temperature sensors within the “isocaloric” chamber can determine the temperature of the fluid within the chamber, relay that information back to the microcontroller, which subsequently adjusts the valve to regulate hot or cold fluid ingress to reduce heat buildup in the ear canal.

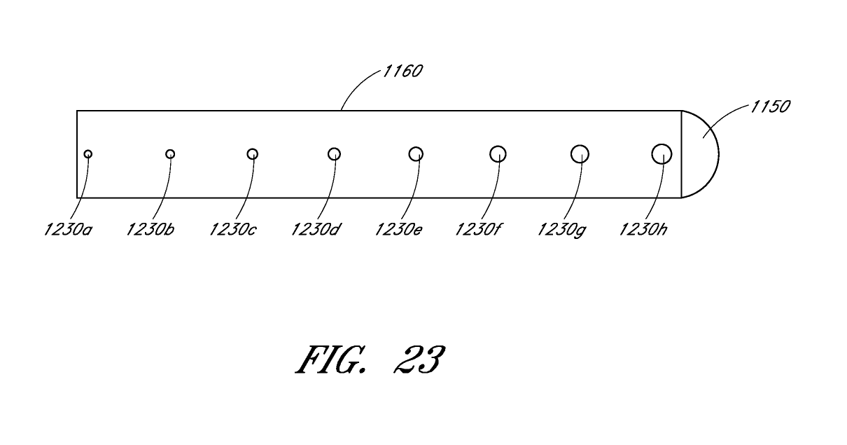

FIG. 23 shows an enlarged view of one embodiment of the fluid ingress probe 1160 with a pressure sensor 1150 at its tip. In several embodiments, the probe comprises a plurality of orifices 1230a-1230h along the length (or a portion of the length) of the probe. In several embodiments, the diameter of the orifices increases as the orifice is positioned more and more distally. In several embodiments, the increased diameter accounts for pressure drop along the length of the tube and ensures equalized fluid flow to the so the proximal, mid, and distal portions of the probe (and hence ejection of the same amount of fluid and equivalent decolonization). The fluid ingress probe may have a silicone surface and/or tip, or other material selected for patient comfort.

The pressure sensor 1150, depending on the embodiments, has one or more functions. First, in some embodiments, in conjunction with the microcontroller, the pressure sensor limits the peak fluid pressure within the ear canal to a safe level, even if fluid outflow is restricted or blocked. In several embodiments the sensor determines the level of fluid in the in the fluid chamber 1170. This data is provided to the microcontroller so it can regulate the egress pump speed to maintain a relatively constant fluid level, thereby preventing fluid from rising too high (though if it does, it overflows out of the vents), or falling too low and leading to “slurping” and possible loss of egress pump prime if the bottom of the egress tube sucks in air instead of fluid. In several embodiments, pressure be sensed by integrating conductors into the egress tube (having their conductive elements exposed at desired levels along the egress tube) and electrically sensing resistance between the sensors. Further, the pressure sensor may detect plugging of the outflow. Outflow plugging is detected, in several embodiments, by the pressure sensor detecting a pressure exceeding a pre-determined threshold and/or by detecting a pressure rise with a slope suggesting that a partial or total outflow occlusion has occurred. Because a small reduction in the patency of the outflow passage will result in a disproportionately large drop in outflow, significant pressure elevations can result. Thus, in several embodiments, the pressure sensor and microcontroller work in concert to adjust (e.g., slow flow or stop flow) the ingress pump. In several embodiments, the vents 1120 serves as a fail-safe backup for pressure release.

When an example device is used to treat colonization or infection, the treatment cycle may be short enough so one phase of heating would be immediately followed by one phase of cooling to reach the isocaloric point where the cooling cycle removes as much heat as was transferred into the patient. When the device is used to remove significant amounts of earwax or pus, alternating hot and cold cycles can isocalorically neutralize the thermal effect upon endolymph density and hence avoid or greatly minimize side effects.

In several embodiments, use of an example device can be preceded by local anesthetic drops to increase patient comfort, especially when treating colonization or infection, which, depending on the embodiment, uses a higher temperature.

In several embodiments, thermal decolonization provides several advantages over antibiotic decolonization. In several embodiments, thermal decolonization reduces the costs of treatment because there is no need to culture the bacteria in advance to identify and select an appropriate antibiotic. Further, thermal decolonization does not promote generation of bacterial resistance and it can be used repeatedly without risking bacterial resistance. Thermal decolonization can also be routinely used to minimize acquiring other infections after exposure (e.g., a healthcare worker inhales air contaminated with germs and can then thermally decolonize the nasal cavity to avoid future colonization). Thermal decolonization can also be used without age restriction, which is not the case for certain antibiotics. Also, there is no risk of allergy or side effects from antibiotics.

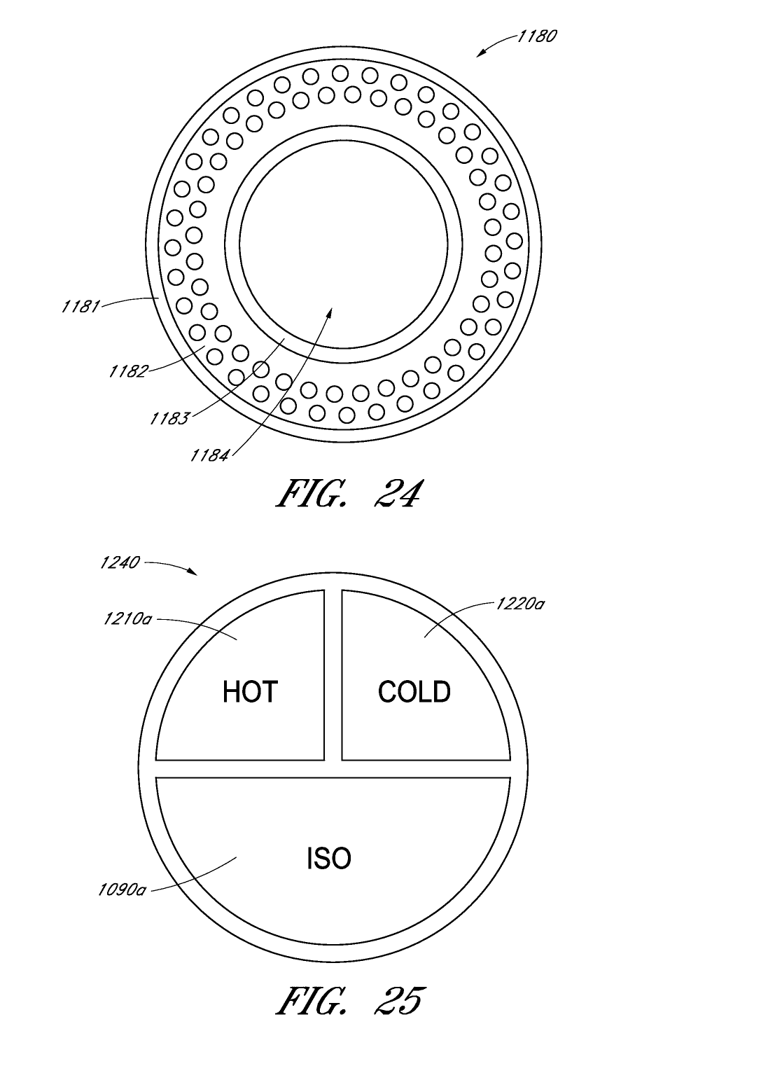

In several embodiments, one or more of the chambers and fluid lines are configured to minimize heat exchange with the environment. For example, in several embodiments, thermal insulation and/or blockers of infrared radiation (e.g., aluminum or some other sheet material including metal or otherwise reflecting foil, such as metalized polymer sheets such as Mylar) are used to reduce heat loss (or gain) while fluid is moved around the device. In several embodiments, the pressure sensor further comprises a temperature sensor to provide precise feedback to the microcontroller. Thermal insulation around the components that do not flex, or flex relatively nominally (e.g., hot, cold, and iso chambers, the valve, and fluid lines external to the patient) comprises, in several embodiments, closed-cell foam or aerogels. In several embodiments, silica aerogels with a carbon soot or titanium dioxide (TiO₂) opacifier are used. In some embodiments, such opaque insulation reduces the need for blockers of infrared radiation. For the flexible portions of the lines, flexible insulation, similar to CRYOGEL® Z, can be applied. In several embodiments, the lines are molded (e.g., of silicone, rubber etc.) with an insulation layer of opacified aerogel spheres embedded in the central portion of the tubing wall, as depicted in FIG. 24. FIG. 24 shows a fluid line 1180 in accordance with several embodiments that has a fluid flow lumen 1184 residing within the interior of the inner wall 1183 of the line. Positioned between the inner wall and outer wall 1181 are insulating materials 1182.

FIG. 25 depicts an example embodiment of a device configuration 1240 (as opposed to the schematic of FIG. 23) with respect to the fluid chambers. The hot chamber 1210a, cold chamber 1220a, and isocaloric chamber 1090a are packaged in a cylindrical formation. Other configurations are also used, in other embodiments (e.g., rectangular or square configurations for space efficiency).

In several embodiments, the valve, fluid ingress pump, and fluid egress pump are located below these fluid chambers so that hot or cold fluid could flow (secondary to gravity) into the fluid ingress pump and automatically prime it. In several embodiments, the vacuum pump is positioned above the fluid chambers and is attached to the ISO chamber via a tube to develop a partial vacuum within it when the device first begins infusing fluid into an ear canal; this partial vacuum draws fluid up (against gravity) the fluid egress line into the fluid egress pump, thereby priming it. Once primed, the vacuum can be turned off for the remainder of the treatment cycle for this patient. In several embodiments, the vacuum is eliminated, as self priming pumps are used. In several embodiments, also positioned above the fluid chambers are the electronics and rechargeable battery (optionally replaced in embodiments of the device that are hard-wired). In several embodiments, the rechargeable battery is inductively charged by a base station for the device. In several embodiments, the base station also comprises a water supply so it could fill the hot and cold chambers (including periodically flushing the chambers, the valve, ingress pump, and ingress lines with hot water to sterilize them). The fluid egress line, egress pump, and isocaloric chamber are sterilized, in several embodiments, by attaching a disposable “U” connector bridging the adjacent ends of the ingress and egress lines and flushing the device. In several embodiments, the “U” connector also serves to keep airborne dust and germs from floating into the fluid lines during storage. Additional sterilization can be achieved by illuminating the chambers with, for example, ultraviolet light. A method for bacterial decolonization of a body cavity of a subject comprises introducing a decolonization fluid into the body cavity, where the decolonization fluid has a temperature sufficient to kill a pathogen in the body cavity. In some examples, the method includes alternating the introduction of the decolonization fluid into the body cavity with an introduction of a cool fluid into the body cavity, the cool fluid having a temperature less than the decolonization fluid, so as to reduce the total heat energy conveyed to the body cavity. For example, a fluid flow (such as a jet, stream, mist, and the like) may alternate or otherwise vary between a decolonization temperature and that of a relatively cool fluid. The cool fluid may have a temperature less than the body temperature of the subject. In some examples, the combination of the cool fluid and decolonization fluid maintains a body portion proximate the body cavity at an approximate body temperature. In some examples, a device is provided that introduces a predetermined volume of decolonization fluid into the body cavity, and then, after a predetermined time interval, removes the predetermined volume of decolonization fluid from the body cavity, optionally followed by the introduction of a predetermined volume of cool fluid and optionally its later removal. This process may be repeated a sufficient number of times to decolonize the body cavity of a pathogen. The subject may be a mammal, such as a human. The body cavity may be an ear cavity, nostril, or other body cavity, or in some examples a crevice such as the gap between toes may be decolonized using described approaches.

In some examples, a device may be used for bacterial decolonization of the oral cavity. The interior of the mouth may provide a refuge for pathogens, such as bacteria, that in some cases may enter the bloodstream during brushing of the teeth. For example, oral anaerobic bacteria have been linked to spinal infections and inflammation. In some examples, a device directs a jet of fluid at a portion of the oral cavity. The temperature of the fluid jet alternates or otherwise varies between a higher temperature that is high enough to kill a pathogen species of interest, such as bacteria, and a lower temperature selected to reduce or eliminate temperature changes in neighboring body structures, such as teeth. In this way, pathogens may be dislodged and killed by a high temperature fluid, and discomfort due to heating of the skin or teeth may be ameliorated through applications of cool fluid at intervals. The fluid may be an aqueous medium, such as water. The fluid may be applied as a stream, jet, mist, or otherwise. The fluid may be thereafter swallowed, spat out, or collected by a return fluid path to an exterior reservoir. The device may include the functionality of a water pik. The higher temperature may be in the range 110° F.-180° F., more particularly in the range 120° F.-150° F. The lower temperature may be in the range 20° F.-90° F., more particularly 40° F.-80° F. In some examples, polarity reversal of a thermoelectric device may be used to modify the heating or cooling of a fluid stream, for example to alternate or otherwise vary the temperature of the fluid stream between higher and lower temperatures. In some examples, apparatus and methods may be adapted for veterinary use, for example being configured to decolonize the nose, ear, or other body cavity of an animal, such as a mammal, for example a dog, cat, horse, and the like. In some example, the decolonization fluid may include further compounds having anti-pathogenic (e.g. antibacterial), pain-reducing, surface tension modifying (e.g. detergents and the like), blockage removing, or other physical, chemical, or physiological effects.

### EXAMPLES

### Example 1

### Nasal Decolonization

Identification of patients in need of nasal decolonization will be determined by cultures and/or rapid staph tests. If need of decolonization is confirmed, the patient will have nasal secretions removed prior to the decolonization procedure. Alternatively, the treatment time will be extended to account for the amount/severity of secretions.

Fluid decolonization media will be heated to the treatment temperature, this example being between 116 and 130 degrees Fahrenheit. An ingress fluid conduit will be placed in the nasal cavity of the subject. A pneumatic sealing balloon on the distal end of the ingress fluid conduit will be inflated to seal the posterior nasal cavity. A nostril cup will be placed on the outer portion of the nostril of the subject.

Heated decolonization media will be infused into the nasal cavity of the subject for a time period sufficient to kill or otherwise inactivate the microorganisms present in the nasal cavity. The sealing balloon will be deflated after treatment, and the apparatus removed.

The subject is optionally cultured after treatment has been completed, in order to determine if additional treatments are required for complete decolonization.

### Example 2

### Otic Decolonization

Identification of patients in need of otic decolonization will be determined by cultures and/or rapid staph tests. If need of decolonization is confirmed, the patient will have otic secretions removed prior to the decolonization procedure. Alternatively, the treatment time will be extended to account for the amount/severity of secretions.

Fluid decolonization media will be heated to the treatment temperature, this example being between 116 and 130 degrees Fahrenheit. An application chamber will be placed over the otic canal of the subject.

Heated decolonization media will be infused into the otic canal of the subject via an ingress tube in the application chamber for a time period sufficient to kill or otherwise inactivate the microorganisms present in the otic canal. Used media will be removed from the canal via an egress tube. Temperature of the used media will be monitored and the temperature of the ingoing media will be adjusted to avoid heat buildup in the ear canal.

The subject is optionally cultured after treatment has been completed, in order to determine if additional treatments are required for complete decolonization.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1.-64. (canceled)

65. A method for bacterial decolonization of a body cavity of a subject, comprising:
contacting a body cavity with heated decolonization fluid, wherein said decolonization fluid is heated to a temperature of between about 110 and about 130 degrees Fahrenheit.

66. The method of claim 65, wherein said body cavity is selected from the group consisting of the anterior portion of the nasal cavity and the inner ear canal.

67. The method of claim 65, wherein said contacting comprises inserting into the body cavity of said subject an elongate treatment member configured for transmission of heated decolonization fluid to a surface of said body cavity, wherein the elongate treatment member comprises a proximal end region in fluid communication with the decolonization fluid, a distal end region configured to deliver a therapeutic amount of the decolonization fluid to the body cavity and a fluid conduit extending between the proximal and distal end regions.

68. The method of claim 65, further comprising positioning a sealing member adjacent the opening of said body cavity in order to prevent unintended loss of decolonization fluid from said body cavity.

69. The method of claim 65, further comprising positioning a sealing member positioned within said body cavity, wherein said sealing portion prevents unintended translocation of said decolonization fluid from said body cavity to an adjacent portion of the body.

70. The method of claim 69, wherein said body cavity comprises the anterior portion of the nasal cavity and said sealing member prevents translocation of said decolonization fluid to the posterior portion of the nasal cavity.

71. The method of claim 65, wherein said body cavity is colonized with bacteria.

72. The method of claim 71, wherein said bacteria are non-endogenous bacteria.

73. The method of claim 71, wherein said bacteria are resistant to antibiotics.

74. The method of claim 73, wherein said antibiotic resistant bacteria comprise methicillin-resistant Staphylococcus aureus.

75. The method of claim 65, wherein said decolonization fluid is a liquid.

76. The method of claim 75, wherein said liquid is water or an aqueous solution.

77. The method of claim 65, further comprising heating decolonization fluid to generate said heated decolonization fluid, wherein said heating is performed by exposing the decolonization fluid to a heating element.

78. The method of claim 77, wherein said heating element heats the decolonization fluid by one or more of direct heating, inductive heating, infrared heating, halogen heating, and/or microwave heating.

79. The method of claim 67, further comprising removing decolonization fluid from the body cavity to a fluid receptacle, wherein the removed fluid is carried by a second fluid conduit in said treatment member.

80. The method of claim 67, further comprising detecting a temperature of the heated decolonization fluid via a temperature sensor at the distal end region of the treatment member.

81. The method of claim 68, wherein the sealing member comprises a nostril cup and the body cavity comprises the nasal cavity.

82. The method of claim 68, wherein the sealing member comprises an inflatable balloon, wherein the body cavity comprises the nasal cavity, and wherein the method further comprises inflating the balloon to separate the anterior portion of the intranasal cavity, which is subject to thermal decolonization, from a posterior portion of the intranasal cavity.

83. The method of claim 65, wherein the body cavity comprises the inner ear canal and contacting the inner ear canal comprises passing said heated decolonization fluid through a treatment member comprising a proximal end region in fluid communication with the heated decolonization fluid, a distal end region configured to deliver a therapeutic amount of the heated decolonization fluid to the body cavity, and a fluid conduit extending between the proximal and distal end regions, wherein the fluid conduit comprises a proximal to distal fluid flow pathway and a distal to proximal fluid flow pathway.

84. The method of claim 83, further comprising positioning a sealing portion outside said ear canal, the sealing portion comprising a chamber through which said fluid conduit passes, the chamber comprising a distally positioned conformable gasket for sealing around the external portion of the ear canal, and a proximally positioned fail-safe vent configured to release decolonization fluid that exceeds a flow capacity of the fluid conduit.

85. The method of claim 83, further comprising contacting the inner ear canal with cooled decolonization fluid alternately with said heated decolonization fluid, wherein said alternating application removes approximately as much heat as was transferred to the ear canal of the subject by contacting the ear canal with the heated decolonization fluid.
